(19) United States
(12) Patent Application Publication
(10) Pub. No.: US 2026/0275411 A1
(43) Pub. Date: Sep. 17, 2026

(54) LINKERS FOR DUPLEX SEQUENCING

(71) Applicant: KEYGENE N.V., Wageningen (NL)

(72) Inventors: René Cornelis Josephus HOGERS, Wageningen (NL); Antonette Johanna Maria VERSTEGE, Wageningen (NL)

(73) Assignee: KEYGENE N.V., Wageningen (NL)

(21) Appl. No.: 19/168,363

(22) PCT Filed: Apr. 4, 2024
(86) PCT No.: PCT/EP2024/059238

§ 371 Date: Sep. 24, 2025

(30) Foreign Application Priority Data
Apr. 4, 2023 (EP) 23166527.4

(51) Int. Cl. C12Q 1/6869

(52) U.S. Cl. CPC C12Q 1/6869

(57) ABSTRACT

The invention pertains to a method for increasing the accuracy of sequencing, in particular ONT sequencing. The invention relates to a method for determining a sequence of interest in a double-stranded nucleic acid molecule, wherein the method comprises the steps of providing a sample comprising the double-stranded nucleic acid molecule, covalently closing at least one end of the double-stranded nucleic acid molecule to provide a double-stranded nucleic acid molecule having one open end and one closed end, sequencing both strands of at least part of the double-stranded nucleic acid molecule in a single sequencing reaction to generate a duplex read; and generating a consensus sequence from the duplex read to determine the sequence in the double-stranded nucleic acid molecule. Preferably, the sequencing is nanopore sequencing.

### FIELD OF THE INVENTION

The present invention is in the field of molecular biology, more in particular in the field of genomics and genetic research. Particularly, the invention is in the field of (deep-) sequencing. Disclosed are new methods and means for improving the accuracy of deep-sequencing, for library preparation, and for complexity reduction of nucleic acid samples.

### BACKGROUND OF THE INVENTION

A significant component of genetic research is the sequence analysis of known and unknown nucleotide sequences. Current sequencing methods are however hampered by their ability to accurately determine the sequence of a nucleic acid molecule. For example, for genotyping and SNP calling it is essential to be able to distinguish between a genuine nucleotide (variant) and a sequencing artifact, especially in case of a low read coverage.

Long read and amplification free sequencing platforms like nanopore sequencing are emerging. Especially for e.g. whole-genome sequencing and mapping, longer reads are highly preferred. It has become evident that long sequence reads significantly improve the quality of de novo genome assemblies, especially for those species harboring large and/or complex genomes. Moreover, long-read sequencing technologies hold the potential to resolve long repeats, polyploidy, and haplotypes and facilitate the identification of genetic elements associated with complex traits through copy number variation (CNV) detection.

The Nanopore sequencing technology platforms are able to produce reads of hundreds of kilobases in size with the potential to go even beyond megabase-sized read lengths. The platform relies on passing of single strands of nucleic acid molecules through a small protein channel (nanopore) that is embedded in an electrically resistant membrane. Single molecules entering the nanopore cause characteristic disruptions in the current. Measuring this disruption, DNA or RNA molecules can be characterized.

The PacBio Single-molecule sequencing technology is able to produce reads up to 25 kb in length with high accuracy in which both strands of a single (circular) dsDNA molecule are read multiple times. The platform relies on detection or incorporation of fluorescent labelled nucleotides of which the duration of the incorporation is recorded and measured. The duration of the incorporation and the fluorescent signal is used to determine which nucleotide is incorporated and (if present) which modification the nucleotide contains. As the original DNA strand is measured, no amplification errors are influencing the accuracy of the resulting sequence.

Element Biosciences Avidity and MGI are examples of short read sequencing platforms. The Element Biosciences Avidity base chemistry binds a circular library molecule to a low-binding surface where the molecule is amplified using a rolling circle reaction. Sequencing is based on sequencing by synthesis where incorporated nucleotides are labelled with fluorescence after the DNA strand is elongated.

The MGI DNBSEQ platforms utilize the DNBSEQ technology in which a sequencing library molecule is circularized via ligation of the two ends of the DNA strand using a connecting oligo which subsequently is used to amplify the circularized molecule via Rolling Circle Amplification (RCA). The generated products are called DNA Nanoballs (DNBs), which are bound to a patterned array. The patterned array with the DNBs is used as input for sequencing using the Combinatorial Probe-Anchor Synthesis (cPAS) Technology, which encompasses primer hybridisation to the adapter region of the DNB and incorporation of fluorescently labelled dNTP probes, washing and imaging.

There is still a need in the art to improve the sequencing read accuracy, in particular to improve the accuracy of long read and/or amplification free sequencing. There is particularly a need in the art to improve the read sequencing accuracy of nanopore sequencing technologies.

### SUMMARY

The invention can be summarized by the following embodiments:
Embodiment 1. An at least partly double-stranded adapter, wherein the adapter comprises an open end and a closed end, and wherein the closed end is covalently closed by a linker, wherein said linker preferably does not consist of deoxyribonucleotides.
Embodiment 2. An adapter according to embodiment 1, wherein the linker comprises a moiety of general formula (L1), with 5′ and 3′ used to represent sites of attachment to the nucleotides forming the end of the adapter:

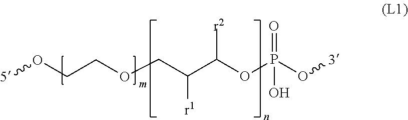

wherein
m is 0, 1, 2, 3, 4, 5, 6, 7, 8, 9, or 10;
n is 0 or 1;
r¹ and r² are each H, or together form a bridging moiety —(O)₀₋₁—(CH₂)₁₋₆—,
wherein preferably when n is 1, m is 0, or when m is not 0, n is 0,
and wherein preferably the linker is selected from the group consisting of C3 Spacer (m is 0, n is 1, r¹ is H and r² is H), Spacer 9 (m is 3 and n is 0), Spacer 18 (m is 6 and n is 0), or one or more 1′,2′-dideoxyriboses (m is 0, n is 1 and r¹ and r² together form bridging moiety —O—(CH₂)₂—).
Embodiment 3. The adapter according to embodiment 1 or 2, wherein the adapter comprises a staggered open end.
Embodiment 4. The adapter according to any one of the preceding embodiments, wherein the adapter is formed by a single-stranded nucleic acid molecule comprising the linker.
Embodiment 5. The adapter according to any one of the preceding embodiments, wherein the adapter comprises an identifier sequence.
Embodiment 6. A method for determining a sequence of interest in a double-stranded nucleic acid molecule, wherein the method comprises the steps of:
a) providing a sample comprising the double-stranded nucleic acid molecule and an adapter according to any one of embodiment 1-5;
b) ligating the adapter to an end of the double-stranded nucleic acid molecule, thereby generating a double-stranded nucleic acid molecule having one open end and one closed end;
c) sequencing both strands of at least part of the double-stranded nucleic acid molecule in a single sequencing reaction to generate a duplex read, wherein preferably the sequencing is performed on an amplification free sequencing platform; and
d) generating a consensus sequence from the duplex read to determine the sequence in the double-stranded nucleic acid molecule.
Embodiment 7. The method according to embodiment 6, wherein step b) comprises the (sub-) steps of:
   b1) ligating the adapter to both ends of the double-stranded nucleic acid molecule, thereby closing both ends of the nucleic acid molecule;
   b2) optionally exposing the sample to an exonuclease; and
   b3) cleaving the closed double-stranded nucleic acid molecule, thereby generating the double-stranded nucleic acid molecule having one open end and one closed end.
Embodiment 8. The method according to embodiment 6 or 7, wherein the nucleic acid molecule is provided by fragmentation of a longer nucleic acid molecule, wherein the longer nucleic acid molecule is preferably a genomic nucleic acid molecule.
Embodiment 9. The method according to embodiment 8, wherein the fragmentation is performed by restriction endonuclease and/or site-directed endonuclease digestion.
Embodiment 10. The method according to embodiment 9, wherein the restriction enzyme and/or site-directed endonuclease creates a single-stranded overhang to the double-stranded nucleic acid molecule.
Embodiment 11. The method according to any one of embodiments 6-10, wherein the nucleic acid molecule provided in step a) comprises a single-stranded overhang and wherein the adapter comprises an overhang that can be ligated to the overhang of the nucleic acid molecule.
Embodiment 12. The method according to any one of embodiments 7-11, wherein the closed double-stranded nucleic acid molecule in step b3) is cleaved by a site-directed endonuclease or a restriction endonuclease, wherein preferably the site-directed endonuclease is an RNA-guided CRISPR nuclease or a TALENs.
Embodiment 13. A method according to any one of embodiments 6-12, wherein the method is performed for a plurality of samples, and wherein preferably the plurality of samples are pooled prior to step c).
Embodiment 14. A method according to any one of embodiments 6-13, wherein a sequencing adapter is ligated to the open end of the double-stranded nucleic acid after step b) and prior to step c).
Embodiment 15. A method according to any one of embodiments 6-14, wherein the amplification free sequencing platform of step c) is nanopore sequencing, preferably nanopore selective sequencing.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al. Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. Thus, for example, reference to “a cell” includes a combination of two or more cells, and the like.

As used herein, the term “about” is used to describe and account for small variations. For example, the term can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

As used herein, the term “adapter” is a single-stranded, double-stranded, partly double-stranded, Y-shaped or hairpin nucleic acid molecule that can be attached, preferably ligated, to the end of other nucleic acids, e.g., to one or both strands of a double-stranded DNA molecule, and preferably has a limited length, e.g., about 10 to about 200, or about 10 to about 100 base pairs, or about 10 to about 80, or about 10 to about 50, or about 10 to about 30 base pairs in length, and is 20 preferably chemically synthesized. The double-stranded structure of the adapter may be formed by two distinct oligonucleotide molecules that are at least partly base paired with one another, or by a single, optionally modified, oligonucleotide strand. Optionally the single oligonucleotide strand is modified to comprise a linker. Preferably, at least one end of the adapter is designed to be compatible with the end of a nucleic acid. The compatible end can be a blunt end or a single-stranded overhang. The attachable end of an adapter may be designed to be compatible with, and optionally ligatable to, overhangs made by cleavage by a restriction enzyme and/or site-directed nuclease, may be designed to be compatible with an overhang created after addition of a non-template elongation reaction (e.g., 3′-A addition).

“And/or”: the term “and/or” refers to a situation wherein one or more of the stated cases may occur, alone or in combination with at least one of the stated cases, up to with all of the stated cases.

“Amplification” used in reference to a nucleic acid or nucleic acid reactions, refers to in vitro methods of making copies of a particular nucleic acid, such as a target nucleic acid, or a tagged nucleic acid. Numerous methods of amplifying nucleic acids are known in the art, and amplification reactions include polymerase chain reactions, ligase chain reactions, strand displacement amplification reactions, rolling circle amplification reactions, transcription-mediated amplification methods such as NASBA (e.g., U.S. Pat. No. 5,409,818), loop mediated amplification methods (e.g., “LAMP” amplification using loop-forming sequences, e.g., as described in U.S. Pat. No. 6,410,278) and isothermal amplification reactions. The nucleic acid that is amplified can be DNA comprising, consisting of, or derived from DNA or RNA or a mixture of DNA and RNA, including modified DNA and/or RNA. The products resulting from amplification of a nucleic acid molecule or molecules (i.e., “amplification products”), whether the starting nucleic acid is DNA, RNA or both, can be either DNA or RNA, or a mixture of both DNA and RNA nucleosides or nucleotides, or they can comprise modified DNA or RNA nucleosides or nucleotides.

A “copy” can be, but is not limited to, a sequence having full sequence complementarity or full sequence identity to a particular sequence. Alternatively, a copy does not necessarily have perfect sequence complementarity or identity to this particular sequence, e.g. a certain degree of sequence variation is allowed. For example, copies can include nucleotide analogs such as deoxyinosine or deoxyuridine, intentional sequence alterations (such as sequence alterations introduced through a primer comprising a sequence that is hybridizable, but not complementary, to a particular sequence), and/or sequence errors that occur during amplification.

The term “complementarity” is herein defined as the sequence identity of a sequence to a fully complementary strand (e.g. the second, or reverse, strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Comprising”: this term is construed as being inclusive and open ended, and not exclusive. Specifically, the term and variations thereof mean the specified features, steps or components are included. These terms are not to be interpreted to exclude the presence of other features, steps or components.

“Construct” or “nucleic acid construct” or “vector”: this refers to a man-made nucleic acid molecule resulting from the use of recombinant DNA technology and which can be used to deliver exogenous DNA into a host cell, often with the purpose of expression in the host cell of a DNA region comprised on the construct. The vector backbone of a construct may for example be a plasmid into which a (chimeric) gene is integrated or, if a suitable transcription regulatory sequence is already present (for example a (inducible) promoter), only a desired nucleotide sequence (e.g., a coding sequence) is integrated downstream of the transcription regulatory sequence. Vectors may comprise further genetic elements to facilitate their use in molecular cloning, such as e.g., selectable markers, multiple cloning sites and the like.

The terms “double-stranded” and “duplex” as used herein, describes two complementary sequences that are base-paired, i.e., hybridized together. “Double-stranded” may be the result of base pairing of two complementary polynucleotides, or due to the base pairing of two complementary sequences within a single (modified) polynucleotide strand. Complementary nucleotide strands are also known in the art as reverse-complement. The two complementary strands may also be described herein as a forward and reverse strand, or a first and a second strand.

The term “effective amount” as used herein, refers to an amount of a biologically active agent that is sufficient to elicit a desired biological effect. All enzymes used in methods disclosed herein are to be used in effective amounts. For example, in some embodiments, an effective amount of an exonuclease refers to the amount of the exonuclease that is sufficient to induce cleavage, for instance of an unprotected or “open-ended” nucleic acid. As will be appreciated by the skilled artisan, the effective amount of an agent may vary depending on various factors such as the agent being used, the conditions wherein the agent is used, and the desired biological effect, e.g. degree of nuclease cleavage to be detected.

“Exemplary”: this terms means “serving as an example, instance, or illustration,” and should not be construed as excluding other configurations disclosed herein.

“Expression”: this refers to the process wherein a DNA region, which is operably linked to appropriate regulatory regions, particularly a promoter, is transcribed into an RNA, which in turn can be translated into a protein or peptide.

A “guide sequence” is to be understood herein as a sequence that directs an RNA or DNA guided endonuclease to a specific site in an RNA or DNA molecule. In the context of a gRNA-CAS complex, “guide sequence” is further to be understood herein as the section of the sgRNA or crRNA, which is required for targeting a gRNA-CAS complex to a specific site in a duplex DNA.

A gRNA-CAS complex is to be understood herein a CAS protein, also named a CRISPR-endonuclease or CRISPR-nuclease, which is complexed or hybridized to a guide RNA, wherein the guide RNA may be a crRNA, a combination of a crRNA and a tracrRNA, or a sgRNA.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The term “nucleotide” includes, but is not limited to, naturally-occurring nucleotides, including deoxyribonucleotides and ribonucleotides, i.e. the building blocks of DNA and RNA. The fundamental structure of a nucleotide is a nitrogenous (purine or pyrimidine) base, a pentose sugar and a phosphate group. The nitrogenous base (or nucleobase) may be guanine, cytosine, adenine, thymine and uracil (G, C, A, T and U, respectively). The term “nucleotide” is further intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “nucleic acid”, “polynucleotide” and “nucleic acid molecule” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein). The nucleic acid may hybridize with naturally occurring nucleic acids in a sequence specific manner analogous to that of two naturally occurring nucleic acids, e.g., can participate in Watson-Crick base pairing interactions. In addition, nucleic acids and polynucleotides may be isolated (and optionally subsequently fragmented) from cells, tissues and/or bodily fluids. The nucleic acid can be e.g. genomic DNA (gDNA), mitochondrial, cell free DNA (cfDNA), DNA from a library and/or RNA from a library.

The term “nucleic acid sample” or “sample comprising a nucleic acid” as used herein denotes any sample containing a nucleic acid, wherein a sample relates to a material or mixture of materials, typically, although not necessarily, in liquid form, containing one or more double-stranded nucleic acid molecules. At least one of the nucleic acid molecules may comprise a sequence of interest. The nucleic acid sample used as starting material in the method of the invention can be from any source, e.g., a whole genome, a collection of chromosomes, a single chromosome, one or more regions from one or more chromosomes, a transcriptome or a selection of transcribed genes, and may be purified directly from the biological source or from a laboratory source, e.g., a processed or chemically modified nucleic acid. The nucleic acid samples can be obtained from the same individual, which can be a human or other species (e.g., plant, bacteria, fungi, algae, archaea, etc.), or from different individuals of the same species, or different individuals of different species. For example, the nucleic acid samples may be from a cell, tissue, biopsy, bodily fluid, genome DNA library, cDNA library and/or a RNA library.

The term “sequence of interest” is to be understood herein as a nucleotide sequence to be analyzed or sequenced, i.e. for which the order of nucleotides is to be assessed by a sequencing method. Known sequencing methods that can be used in the method of the present invention are, e.g. nanopore sequencing, PacBio Single-molecule sequencing, MGI DNBSEQ and Element Biosciences Avidity sequencing. A preferred sequencing method is an amplification free sequencing method, wherein preferably the amplification free sequencing is at least one of nanopore sequencing and PacBio single-molecule sequencing. A preferred sequencing method is nanopore sequencing. Alternatively or in addition, the sequencing method may provide epigenetic information about the sequence of interest. A sequence of interest includes, but is not limited to, any genetic sequence preferably present within a cell, such as, for example (part of) a chromosome, (part of) a gene, or a non-coding sequence within or adjacent to a gene. The sequence of interest may be any sequence within a nucleic acid sample, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. A sequence of interest is preferably the object of a further analysis or action, such as, but not limited to copying, amplification, sequencing and/or other procedure for nucleic acid interrogation. Preferably, the sequence of interest is a small or longer contiguous stretch of nucleotides (i.e. a polynucleotide) of a single strand of duplex DNA, wherein said duplex DNA further comprises a complementary strand comprising a sequence complementary to the sequence of interest. Preferably, said duplex DNA is genomic DNA (gDNA) and/or cell free DNA (cfDNA). The sequence of interest may be present in a chromosome, an episome, a transcript, an organellar genome such as mitochondrial or chloroplast genome or genetic material that can exist independently to the main body of genetic material such as an infecting viral genome, plasmids, episomes, transposons for example. A sequence of interest may be within the coding sequence of a gene or within a transcribed non-coding sequence such as, for example, within the 5′ untranslated region, the 3′ untranslated region or intron. Said nucleic acid sequence of interest may be present in a double or a single strand nucleic acid. The sequence of interest can be, but is not limited to, a sequence having or suspected of having, a polymorphism, e.g. a SNP. A sequence of interest can be a sequence unknown in the art, e.g. de novo sequencing.

The term “oligonucleotide” as used herein denotes a single-stranded multimer of nucleotides, preferably of about 2 to 200 nucleotides, or up to 500 nucleotides in length. Oligonucleotides may be synthetic or may be made enzymatically, and, in some embodiments, are about 10 to 50 nucleotides in length. Oligonucleotides may contain ribonucleotide monomers (i.e., may be oligoribonucleotides) or deoxyribonucleotide monomers. An oligonucleotide may be about 10 to 20, 20 to 30, 30 to 40, 40 to 50, 50 to 60, 60 to 70, 70 to 80, 80 to 100, 100 to 150, 150 to 200, or about 200 to 250 nucleotides in length, for example.

“Plant” refers to either the whole plant or to parts of a plant, such as tissue or organs (e.g. pollen, seeds, gametes, roots, leaves, flowers, flower buds, anthers, fruit, etc.) obtainable from the plant, as well as derivatives of any of these and progeny derived from such a plant by selfing or crossing. Non-limiting examples of plants include crop plants and cultivated plants, such as African eggplant, alliums, artichoke, asparagus, barley, beet, bell pepper, bitter gourd, bladder cherry, bottle gourd, cabbage, canola, carrot, cassava, cauliflower, celery, chicory, common bean, corn salad, cotton, cucumber, eggplant, endive, fennel, gherkin, grape, hot pepper, lettuce, maize, melon, oilseed rape, okra, parsley, parsnip, pepino, pepper, potato, pumpkin, radish, rice, ridge gourd, rocket, rye, snake gourd, sorghum, spinach, sponge gourd, squash, sugar beet, sugar cane, sunflower, tomatillo, tomato, tomato rootstock, vegetable Brassica, watermelon, wax gourd, wheat and zucchini.

“Plant cell(s)” include protoplasts, gametes, suspension cultures, microspores, pollen grains, etc., either in isolation or within a tissue, organ or organism. The plant cell can e.g. be part of a multicellular structure, such as a callus, meristem, plant organ or an explant.

“Primer” refers to a single stranded synthetic nucleotide molecule which can prime the synthesis of DNA. A DNA polymerase cannot synthesize DNA de novo without a primer: it can only extend an existing DNA strand in a reaction wherein the complementary strand is used as a template to direct the order of nucleotides to be assembled. From the 3′-end of a primer hybridized to the complementary DNA strand, nucleotides are incorporated using the complementary strand as a template. Primers can be amplification primers used in an amplification reaction including, but not limited to, a polymerase chain reaction (PCR), or sequence primers used to sequence DNA.

The “protospacer sequence” is the sequence that is recognized or hybridizable to a guide sequence within a guide RNA, more specifically the crRNA or, in case of a sgRNA, the crRNA part of the sgRNA.

An “endonuclease” is an enzyme that hydrolyses at least one strand of a duplex DNA or a strand of an RNA molecule, upon binding to its target or recognition site. An endonuclease is to be understood herein as a site-directed endonuclease and the terms “endonuclease” and “nuclease” are used interchangeable herein. A restriction endonuclease is to be understood herein as an endonuclease that hydrolyses both strands of the duplex at the same time to introduce a double strand break in the DNA. A “nicking” endonuclease is an endonuclease that hydrolyses only one strand of the duplex to produce DNA molecules that are “nicked” rather than cleaved.

An “exonuclease” is defined herein as any enzyme that cleaves one or more nucleotides from the (open) end (exo) of a polynucleotide.

“Reducing complexity” or “complexity reduction” is to be understood herein as the reduction of a complex nucleic acid sample, such as samples derived from genomic DNA, cfDNA derived from liquid biopsies, isolated RNA samples and the like. Reduction of complexity results in the enrichment of one or more specific nucleic acids, preferably comprising a sequence of interest, comprised within the complex starting material and/or the generation of a subset of the sample, wherein the subset comprises or consists of one or more specific nucleic acids, preferably comprising a sequence of interest, comprised within the complex starting material, while non-specific nucleic acids, preferably not comprising a sequence of interest, are reduced in amount by at least 20%, 30%, 40%, 50%, 60%, 70%, 80%, 90%, 91%, 92%, 93%, 94%, 95%, 96%, 97%, 98% or 99% as compared to the amount of non-specific nucleic acids in the starting material, i.e. before complexity reduction.

Reduction of complexity is in general performed prior to further analysis or method steps, such as amplification, barcoding, sequencing, determining epigenetic variation etc. Preferably complexity reduction is reproducible complexity reduction, which means that when the same sample is reduced in complexity using the same method, the same, or at least comparable, subset is obtained, as opposed to random complexity reduction.

Examples of complexity reduction methods include for example AFLP® (Keygene N.V., the Netherlands; see e.g., EP 0 534 858), Arbitrarily Primed PCR amplification, capture-probe hybridization, the methods described by Dong (see e.g., WO 03/012118, WO 00/24939) and indexed linking (Unrau P. and Deugau K. V. (1994) Gene 145:163-169), the methods described in WO2006/137733; WO2007/037678; WO2007/073165; WO2007/073171, US 2005/260628, WO 03/010328, US 2004/10153, genome portioning (see e.g. WO 2004/022758), Serial Analysis of Gene Expression (SAGE; see e.g. Velculescu et al., 1995, see above, and Matsumura et al., 1999, The Plant Journal, vol. 20 (6): 719-726) and modifications of SAGE (see e.g. Powell, 1998, Nucleic Acids Research, vol. 26 (14): 3445-3446; and Kenzelmann and Mühlemann, 1999, Nucleic Acids Research, vol. 27 (3): 917-918), MicroSAGE (see e.g. Datson et al., 1999, Nucleic Acids Research, vol. 27 (5): 1300-1307), Massively Parallel Signature Sequencing (MPSS; see e.g. Brenner et al., 2000, Nature Biotechnology, vol. 18:630-634 and Brenner et al., 2000, PNAS, vol. 97 (4): 1665-1670), self-subtracted cDNA libraries (Laveder et al., 2002, Nucleic Acids Research, vol. 30 (9): e38), Real-Time Multiplex Ligation-dependent Probe Amplification (RT-MLPA; see e.g. Eldering et al., 2003, vol. 31 (23): el53), High Coverage Expression Profiling (HiCEP; see e.g. Fukumura et al., 2003, Nucleic Acids Research, vol. 31 (16): e94), a universal micro-array system as disclosed in Roth et al. (Roth et al., 2004, Nature Biotechnology, vol. 22 (4): 418-426), a transcriptome subtraction method (see e.g. Li et al., Nucleic Acids Research, vol. 33 (16): el36), and fragment display (see e.g. Metsis et al., 2004, Nucleic Acids Research, vol. 32 (16): el27).

“Sequence” or “Nucleotide sequence”: This refers to the order of nucleotides of, or within a nucleic acid. In other words, any order of nucleotides in a nucleic acid may be referred to as a sequence or nucleic acid sequence. For example, the target sequence is an order of nucleotides comprised in a single strand of a DNA duplex.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide are obtained. Sequencing is preferably performed by an amplification free sequencing method, preferably at least one of nanopore sequencing and PacBio single-molecule sequencing. Sequencing is preferably performed by nanopore sequencing methods, such as those commercialized by Oxford Nanopore Technologies (ONT), or electronic-detection based methods such as lon Torrent technology commercialized by Life Technologies. Preferably, the next-generation sequencing method is a nanopore sequencing method, preferably a nanopore selective sequencing method.

“Nanopore selective sequencing” is to be understood herein as selective sequencing of single molecules in real time using nanopore sequencing technology such as from Oxford Nanopore or Ontera, and mapping streaming nanopore current signals or base calls to a reference sequence in order to reject non-target sequences. In response to the data being generated, the sequencer is steered to either pursue sequencing of a nucleic acid, or to quit and remove the nucleic acid from the sequencing pore by reversing the polarity of the voltage across the specific pore for a certain short period of time sufficient to eject the non-target molecule and make the nanopore available for a new sequencing read. Examples of Nanopore selective sequencing methods are described in Payne et al., 2020 (Nanopore adaptive sequencing for mixed samples, whole exome capture and targeted panels, Feb. 3, 2020; DOI: 10.1101/2020.02.03.926956) and Kovaka et al. 2020 (Targeted nanopore sequencing by real-time mapping of raw electrical signal with UNCALLED, Feb. 3, 2020; doi: 10.1101/2020.02.03.931923), which are incorporated herein by reference.

A “double-stranded nucleic acid molecule” in the context of the invention may be a small or longer stretch, or selected portion of a nucleic acid. Prior to the performing the method of the invention, the double-stranded nucleic acid molecule may be comprised within a larger nucleic acid molecule, e.g. within a larger nucleic acid molecule present in a sample to be analysed. Preferably, the double-stranded nucleic acid molecule comprises a sequence of interest.

A “target sequence” is defined herein as a sequence present in the double-stranded acid molecule as defined herein, which sequence is recognized by at least one of a nuclease and nickase as defined herein.

In some aspects, a “plurality” or “set” of nucleic acid molecules used in the method of the invention comprise one or more sequences of interest that are selected to be determined. Optionally, such set consists of structurally or functionally related nucleic acid molecules. A nucleic acid molecule in the context of the invention can comprise both natural and non-natural, artificial, or non-canonical nucleotides including, but not limited to, DNA, RNA, BNA (bridged nucleic acid), LNA (locked nucleic acid), PNA (peptide nucleic acid), morpholino nucleic acid, glycol nucleic acid, threose nucleic acid, epigenetically modified nucleotide such as methylated DNA, and mimetics and combinations thereof.

### DETAILED DESCRIPTION

The inventors discovered a method to increase the sequencing read accuracy, in particular to increase the sequencing read accuracy of long read sequencing using an adapter comprising one open and one closed end. In a first aspect, provided is an adapter, wherein the adapter is at least partly double-stranded and wherein one end of the adapter is covalently closed by a linker. The adapter of the invention is also indicated herein as a “linker-comprising adapter”, a “linked adapter”, “linker adapter” or as an “adapter comprising a closed end”. Preferably, the adapter is for use in a method for determining a sequence of interest in a double-stranded nucleic acid molecule. Preferably, the adapter can be attached to the nucleic acid molecule comprising the sequence of interest.

The double-stranded structure of the adapter is preferably formed by a single-stranded oligonucleotide that is modified to comprise a (n internal) linker, preferably a linker as defined herein. Therefore, also provided is said modified single-stranded oligonucleotide. At least part of the sequence upstream (5′) and downstream (3′) of the linker are complementary sequences, such that the oligonucleotide can fold back into a double-stranded (base-paired) structure, thereby placing the linker at one end of the double stranded structure, which is indicated herein as the “closed end” of the adapter, as opposed to the opposite end of the adapter which is indicated herein as the “open end” of the adapter. The open end of the adapter is preferably suitable for ligation to a nucleic acid molecule comprising a sequence of interest and preferably is 5′-phosphorylated. The complementary sequences flanking the linker can be directly (or “immediately”) flanking the linker. Alternatively, there can be one or more nucleotides located in between the complementary sequences and the linker.

The modified single-stranded oligonucleotide forming the adapter may comprise, in a 5′-end to 3′-end order, the elements A, B, C, D, and E, wherein:
Element C is a linker, preferably a linker as defined herein;
Element B and D are complementary nucleotide sequences (also indicated in the art as “reversed-complement”) of equal length, i.e. together these complementary sequences will form the double-stranded structure of the adapter. Elements B and D may each comprise about 1, 2, 3, 4, 5, 6, 7, 8, 9, 10, 15, 20, 25, 30, 35, 40, 45, 50 or more nucleotides. Elements B and D may each comprise less than about 500, 300, 200, 150, 100, 80, 70, 60, or less than about 50 nucleotides. Preferably elements B and D each comprise about 10-200, 10-100, 10-80, 10-50, or about 10-30 nucleotides. Preferably, elements B and D directly flank linker element C;
Elements A and E are optional elements, preferably comprising or consisting of a sequence of one or more nucleotides. Optionally, elements A and E are absent, rendering a blunt ended adapter. Optionally, either one of the element A or E is present, while the other is absent, rendering an adapter with a 5′-end or 3′-end overhang, respectively, also indicated as a staggered or sticky-ended adapter. The overhang may consist of 1, 2, 3, 4, 5, 6, 7, 8, 9, 11, 12, 13, 14, or 15 nucleotides. Preferably, element A is absent, and element E consist of a thymidine nucleotide, rendering and adapter with a 3′-T overhang.

Preferably, the modified single-stranded oligonucleotide forming the adapter of the invention comprises or consists of the following elements ordered from the 5′ to 3′-end:
B, C and D;
A, B, C and D; or
B, C, D and E.

Preferably these elements are covalently linked. The skilled person readily understands each one of the elements A, B, D and E may comprise functional domains such as an identifier sequence (e.g. a sample identifier, molecular identifier or UMI and/or signatory sequence, as defined herein), primer binding sequence, and/or a restriction element recognition site. Preferably, these functional domains are comprised within the double stranded structure formed by the elements B and D. Preferably, such restriction enzyme recognition site is recognized by an enzyme capable of restricting double stranded DNA.

Preferably the single-stranded oligonucleotide, with the exception of element C, comprises the bases adenine (A), guanine (G), cytosine (C), thymine (T) and/or uracil (U). Optionally, the single-stranded oligonucleotide comprises the bases adenine (A), guanine (G), cytosine (C), and/or thymine (T). The single-stranded oligonucleotide may comprise DNA, RNA or a combination of RNA and DNA. Preferably the single-stranded oligonucleotide, with the exception of element C, consists of DNA, RNA or a combination of RNA and DNA. The oligonucleotide, with the exception of element C, preferably does not comprise at least one of modified DNA, modified RNA, peptide nucleic acid (PNA), locked nucleic acid (LNA), glycerol nucleic acid (GNA), bridged nucleic acid (BNA) and/or threose nucleic acid (TNA). The oligonucleotide, with the exception of element C, preferably consists of DNA, i.e. consists of the deoxyribonucleotides A, G, C and/or T. Optionally, the one or more of the deoxyribonucleotides of the single-stranded oligonucleotide of elements A, B, D or E, preferably element B and/or D, comprise natural modifications such as one or more methylation groups.

Preferably, the sequences directly (or immediately) flanking the linker are complementary sequences. In other words, preferably elements B and D directly flank linker element C as defined herein. Preferably about 10-200, 10-100, 10-80, 10-50, or about 10-30 nucleotides directly upstream and directly downstream the linker are complementary sequences. Preferably, the adapter does not form or comprise a hairpin structure. A hairpin structure is to be understood in the art as a stretch of single stranded (unpaired) nucleotides in between two complementary stretches of nucleotides. Preferably, at least about 45%, 50%, 55%, 60%, 65%, 70%, 75%, 80%, 85%, 90%, 95%, 96%, 97%, 98%, 99%, or about 100% of the nucleotides in the adapter are double-stranded. Preferably, the adapter is a fully double-stranded (base-paired) structure, optionally with the exception of the open end of the adapter. Optionally, the adapter comprises a staggered (or “sticky”) open end (in other words, the adapter may comprise one of the elements A and E). Alternatively, the adapter comprises a blunt open end (in other words, the adapter does not comprise any one of elements A and E). Optionally, the adapter comprises a 3′-T overhang (in other words, the adapter comprises or consists of elements B, C, D and E, wherein element E consists of a single nucleotide that is a thymidine).

Preferably, the open end of the adapter is compatible with an end of the nucleic acid molecule comprising a sequence of interest. The compatible end can be a blunt end or a single-stranded overhang. For example, in case the nucleic acid molecule comprises an 3′-A overhang, the open end of the adapter preferably comprises a 3′-T overhang. Similarly, in case the nucleic acid molecule is obtained by enzyme digestion leaving an overhang of 1, 2, 3, 4, 5 or more nucleotides, the adapter preferably comprises an overhang of respectively 1, 2, 3, 4, 5 or more nucleotides that are complementary to the overhang of the nucleic acid molecule. In other words, preferably the adapter comprises an overhang that is compatible to the end of a restriction fragment created by the use of a restriction enzyme and can be ligated thereto. A restriction fragment is a nucleic acid molecule produced by digestion with a restriction endonuclease. The other (closed) end of the adapter cannot be ligated to a nucleic acid molecule, or to an adapter.

The adapter preferably has a limited length. The length of the adapter is preferably at least sufficient to form a double-stranded structure comprising the linker on one end of the adapter. Preferably the adapter forms a double-stranded structure under conditions, preferably optimal conditions, for annealing and/or ligating the adapter to the nucleic acid molecule comprising a sequence of interest. The length of the double-stranded adapter preferably is at least about 5, 10, 15, 20 or at least about 25 base pairs, Preferably, the length of the double stranded adapter is about 10-200, 10-100, 10-80, 10-50, or about 10-30 base pairs in length. Hence preferably, the modified single-stranded oligonucleotide that forms the double-stranded adapter is about 20-400, 20-160, 20-100 or about 20-60 nucleotides. The linker is preferably located in, or close to, the middle of the modified single-stranded oligonucleotide. Hence preferably in case the single-stranded oligonucleotide comprises n nucleotides, the linker is located at position ½ n. Optionally the linker is located at position ½ n plus x nucleotides, wherein x=1, 2, 3, 4, 5, 6, 7, 8, 9 or 10. Optionally the linker is located at position ½ n minus x nucleotides, wherein x=1, 2, 3, 4, 5, 6, 7, 8, 9 or 10.

Element C may be any linker known in the art. Various types of linkers are commercially available. Preferably, the linker does not consist of deoxyribonucleotides. Preferably, the linker does not consist of nucleotides. The linker preferably does not comprise a nucleobase, wherein “nucleobases” are also indicated in the art as “nitrogenous bases”. Preferably, the linker of the adapter of the invention is a linker that is free of nucleobases. In other words, the linker of the invention does not comprise any one of an adenine, cytosine, guanine, thymine, and uracil. In the context of this description, linkers connect the 3′-OH of one strand of the double-stranded adapter to the 5′-O—P(O)₂—OH of the other strand of the double-stranded adapter. In other words, the linker is positioned within the sugar-phosphate backbone of the single stranded oligonucleotide that can fold back to form the adapter, i.e. between the sugar-phosphate backbones of element B and D. A skilled person knows how to select a suitable linker.

Preferred linkers comprise a moiety of general formula (L1), with 5′ and 3′ used to represent sites of attachment to the nucleotides forming the end of the adapter:

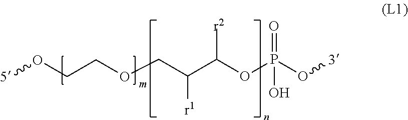

wherein
m is 0, 1, 2, 3, 4, 5, 6, 7, 8, 9, or 10;
n is 0 or 1;
r¹ and r² are each H, or together form a bridging moiety —(O)₀₋₁—(CH₂)₁₋₆—.

Preferably when n is 1, m is 0. Preferably when m is not 0, n is 0. Preferably r¹ and r² are each H. When r¹ and r² together form a bridging moiety —(O)₀₋₁—(CH₂)₁₋₆—, it is preferably —(CH₂)₂₋₄— or —O—(CH₂)₁₋₃, more preferably —(CH₂)₂₋₃— or —O—(CH₂)₁₋₂, most preferably —(CH₂)₃— or —O—(CH₂)₂—, wherein the bridging moieties comprising oxygen are always preferred over their analogues of equal length. Preferably the oxygen, when present, is linked to the position marked r¹.

Further preferred linkers comprise a moiety of general formula (L2) or (L3) or (L4) or (L5) with features as described for (L1):

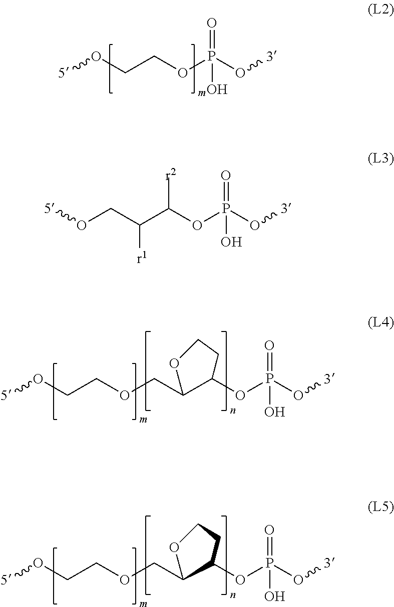

In preferred embodiments the linker comprises or consists of one or more moieties having general formula (L1) selected from the following (with reference names in parentheses):
n is 0 as indicated in general formula (L2), wherein preferably m is 3 (Spacer 9, also indicated herein as /iSpC9/ or triethylene glycol spacer) as indicated in formula (L7), or wherein preferably m is 6 (Spacer 18, also indicated herein as/iSpC18/or hexaethyleneglycol spacer) as indicated in formula (L8);
m is 0; n is 1 as indicated in general formula (L3), wherein preferably r¹ and r² are each H (C3 Spacer, also indicated herein as/iSpC3/or phosphoramidite spacer) as indicated in formula (L6);
n is at least 1; r¹ and r² together form bridging moiety —O—(CH₂)₂— as in formula (L4), preferably as in formula (L5), even more preferably wherein m is 0 and n is 1 (dSpacer, also indicated herein as /idSp/ or 1′,2′-dideoxyribose spacer) as in formula (L9);
   A particularly preferred linker comprises or consists of a moiety of formula (L6), (L7), or (L8) or (L9):

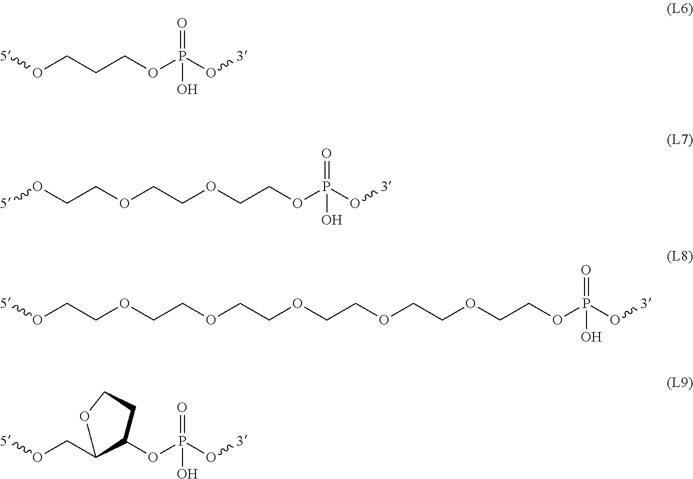

In some embodiments the linker is Spacer 9 (having structural formula (L7)) or Spacer 18 (having structural formula (L8)). In some embodiments the linker is C3 Spacer (having structural formula L6) or dSpacer (having structural formula (L9)). In some embodiments the linker is Spacer 9 or Spacer 18 or C3 Spacer.

Preferably, the linker of the adapter of the invention comprises or consists of at most 6, 5, 4, 3, 2 or 1 moiety(ies) as defined herein. Preferably, the linker comprises or consists of at most 1 moiety of the formula (L2), wherein m is at most 6, 5, 4, 3, 2 or 1. Alternatively or in addition, the linker may comprise or consist of at most 6, 5, 4, 3, 2, or 1 moiety(ies) of the formula (L9).

Linkers can be routinely used to be incorporated into the single-stranded oligonucleotide forming the double-stranded adapter, and a skilled person is aware of which chemistry is suitable for this. For instance, phosphoramidite chemistry can be used, using 4,4′-dimethoxytrityl (DMT) protecting groups when appropriate. Further guidance is available from manuals of commercial linker providers, such as “integrated DNA technologies, Inc.” (Coralville, Iowa, USA). Multiple moieties can be used together to form the linker. Preferably, the linker comprises several consecutive identical or different moieties of any one of the general formula (L1), where each 5′ of the linker (as depicted) is connected to a 3′ (as depicted) of its neighboring linker, such as 1, 2, 3, 4, 5, 6, 7, 8, 9, 10 or more consecutive identical or different moieties of the general formula (L1), preferably 1 or 2 consecutive moieties of the general formula (L1) form the linker.

For instance, two or three or more moieties of (L1) could be consecutively used, where each 5′ of the linker (as depicted) is connected to a 3′ (as depicted) of its neighboring moiety. In some embodiments a single moiety of general formula (L1) is used. In other embodiments 2 consecutive such moieties are used. In this embodiment, the linker may have general formula (L12):

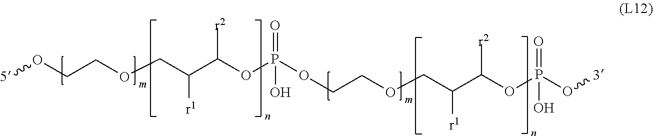

In other embodiments 3 consecutive such moieties are used. In this embodiment, the linker may have general formula (L13):

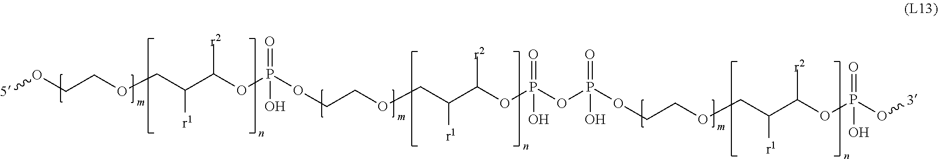

In other embodiments more than 2 consecutive moieties are used, wherein each of the 5′ end of following moiety is linked to the 3′-end of the previous moiety as indicated above for connecting moiety 1 and 2 in (L13). Optionally, 4, 5 or 6 consecutive such moieties are used. Preferably 1, 2, or 3 consecutive such moieties are used. Optionally a single such moiety is used as the linker.

Preferably, linker comprises several consecutive moieties of the general formula (L5), where each 5′ of the linker (as depicted) is connected to a 3′ (as depicted) of its neighboring linker, such as 1, 2, 3, 4, 5, 6, 7, 8, 9, 10 or more consecutive moieties of the general formula (L5), preferably 1 or 2 consecutive moieties of the general formula (L5) form the linker.

The linker may comprise or consists of one or more consecutive dSpacer moieties, e.g. 1, 2, 3, 4, 5, 6, 7, 8, 9, 10 or more dSpacer moieties. Optionally the linker comprises or consists of 1, 3 or 5 dSpacer moieties, i.e. the linker is dSpacer, dSpacer-dSpacer-dSpacer or dSpacer-dSpacer-dSpacer-dSpacer-dSpacer. Preferably, the linker comprises at most 6, 5, 4, 3, 2, or 1 dSpacer moiety(ies). The linker may have structural formula (L10) or (L11), preferably the linker has structural formula (L10):

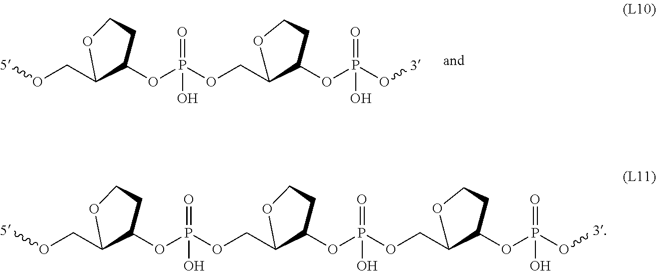

A preferred linker has a structural formula selected from the group consisting of (L6), (L7), (L8), (L9), L10), and (L11) preferably selected from the group consisting of (L6), (L8), (L9) and (L10). The linker preferably has structural formula (L6) or (L10).

Preferably, linker comprises several consecutive moieties of the general formula (L9), where each 5′ of the linker (as depicted) is connected to a 3′ (as depicted) of its neighboring linker, such as 1, 2, 3, 4, 5, 6, 7, 8, 9, 10 or more consecutive moieties of the general formula (L9), preferably 1 or 2 consecutive moieties of the general formula (L9) form the linker. Preferably, the linker has general formula (L9) or (L10).

The adapter of the invention may comprise an identifier sequence or “barcode” or “tag” for tagging the double-stranded nucleic acid molecule. Hence tagging of the double-stranded nucleic acid molecule is preferably achieved by (adapter) hybridization and/or (adapter) ligation. The identifier is preferably at least one of a sample identifier, a unique molecular identifier (UMI) and/or a signatory sequence as defined herein. In addition or alternatively, a further adapter may be attached or ligated to a the nucleic acid molecule to be sequenced. Said further adapter may (also) comprise an identifier sequence. The identifier sequence preferably remains covalently attached to the nucleic acid up to (and including) the step of sequencing. Optionally, the nucleic acid that is sequenced comprises a combination of identifier sequences. For instance, at least one identifier may be introduced by ligation of the adapter of the invention, and at least one identifier may be added to the open end of the nucleic acid, for instance together with introducing a sequencing primer binding site, and/or by attaching a sequencing adapter. In case a Y-shaped adapter is ligated to the open end of the (adapter-comprising) nucleic acid to be sequenced, two identifiers may be present, wherein each identifier is located in a different single-stranded arm of the Y shaped adapter. In combination with a possible identifier introduced by ligation of the adapter of the invention, the nucleic acid comprising the sequence of interest may be labeled with three different identifiers. The function of the identifiers may be different. For instance, the identifier introduced by ligation of the adapter of the invention may be a sample identifier, and the (combination of) identifier(s) introduced at the open end may be molecular identifier(s), or vice versa. Such (combination of) identifier(s) may serve to trace back the template molecule after a process of amplification of the labelled nucleic acid molecules. The UMI may be a separate sequence within the adapter of the invention. The adapter of the invention may comprise a sample identifier as well as a UMI. In addition or alternatively, a UMI and/or sample identifier may also be added to the nucleic acid molecule comprising the sequence of interest prior to ligation of the adapter of the invention. Said UMI and/or sample identifier may be part of a primer (for instance part of the 5′-end overhang of a primer) that is annealed and/or a further adapter (e.g. an “indexing-adapter”) that is ligated to a nucleic acid molecule comprising the sequence of interest, for instance prior to ligation of the adapter of the invention. The adapter of the invention may subsequently be ligated to the further adapter comprising the identifier sequence or to a sequence introduced by the primer and/or the adapter of the invention can be ligated to the other end of the nucleic acid molecule comprising a sequence of interest. Optionally, two indexing-adapters are ligated to the nucleic acid molecule comprising the sequence of interest prior to ligation of the adapter of the invention and prior to ligating a potential sequencing adapter, i.e. one indexing-adapter at either end of said nucleic acid molecule. Preferably, said indexing-adapter is open ended on both sites of the adapter.

A sample identifier may connect the sequence of a nucleic acid molecule to a specific sample. For example, the adapter used in the method provided herein may comprise an identifier sequence that is specific for a certain sample. Each additional sample can be processed using adapters having an identifier sequence specific for said additional sample. The processed samples can subsequently be pooled and the obtained sequences can be assigned to a specific sample using the sample identifier sequence. In a preferred embodiment, multiple samples are treated in parallel by performing steps of ligating the adapter of the invention to nucleic acid molecules present in multiple samples in parallel and pooling the samples prior to sequencing.

A UMI is a substantially unique sequence, tag or barcode, preferably fully unique, that is specific for a nucleic acid molecule, i.e. unique (substantially or fully) for each nucleic acid molecule present in a particular sample. The UMI may have random, pseudo-random or partially random, or non-random nucleotide sequences. A UMI can be used to uniquely identify the originating molecule from which a sequencing read is derived. For example, reads of amplified nucleic acid molecules can be collapsed into a single consensus sequence from each originating nucleic acid molecule. As indicated above, the UMI may be fully or substantially unique. Fully unique is to be understood herein as that every (adapter-ligated) nucleic acid molecule of a sample provided in the method of the invention comprises a unique tag that differs from all the other tags comprised in further (adapter-ligated) nucleic acid molecules used in the method of the invention. Substantially unique is to be understood herein in that each adapter-ligated nucleic acid molecule provided in the method of the invention comprises a random UMI, but a low percentage of these adapter-ligated nucleic acid molecules may comprise the same UMI. Preferably, substantially unique molecular identifiers are used in case the chances of tagging the exact same molecule comprising the same sequence with the same UMI is negligible. Preferably, the UMI is fully unique in relation to a specific sequence of the nucleic acid molecule. The UMI preferably has a sufficient length to ensure this uniqueness. In some implementations, a less unique molecular identifier (i.e. a substantially unique identifier, as indicated above) can be used in conjunction with other identification techniques to ensure that each nucleic acid molecule is uniquely identified during the sequencing process. A UMI can be used to confirm that a sequence read originates from a particular nucleic acid molecule, and can be used to construct consensus sequences for a single original nucleic acid molecule. In addition or alternatively, a UMI can e.g. be used to confirm that certain amplicons are derived from a specific original nucleic acid molecule or fragment. Hence a UMI may be ligated or annealed prior to amplification, wherein said amplification step may take place prior to the ligation of the adapter of the invention to the resulting nucleic acid amplicon molecules or fragments.

An identifier sequence may range in length from about 2-100 nucleotide bases or more, and preferably has a length between about 4-16 nucleotide bases. The identifier sequence can be a consecutive sequence or may be split into several subunits. These subunits may be present in a single adapter and/or primer or may be present in separate adapters and/or primers. For instance, if the nucleic acid molecule to be sequenced is flanked by two adapters, each of these two adapters may comprise a subunit of the identifier sequence. In order to obtain consensus sequences, the sequence reads obtained in the method of the invention may be grouped based on the combining the information of each of the two identifier subunits.

Preferably the identifier sequence does not contain two or more consecutive identical bases. Furthermore, there is preferably a difference between identifier sequences of at least two, preferably at least three bases.

In a further aspect, provided herein is a method for determining a sequence in a double-stranded nucleic acid molecule, preferably of determining a sequence of interest in a double-stranded nucleic acid molecule. Preferably, the method comprises the steps of:
a) providing a sample comprising the double-stranded nucleic acid molecule and an adapter comprising a linker as defined herein;
b) ligating the adapter to an end of the double-stranded nucleic acid molecule, thereby generating a double-stranded nucleic acid molecule having one open end and one closed end;
c) sequencing both strands of at least part of the double-stranded nucleic acid molecule in a single sequencing reaction to generate a duplex read; and
d) generating a consensus sequence from the duplex read to determine the sequence in the double-stranded nucleic acid molecule.

Optionally, the one or more nucleic acids to be sequences are genomic sequences and/or genomes from individuals such as human, plants, animals, insects or microorganisms (e.g. viruses or bacteria). Hence, the method as defined herein may also be considered as e.g. at least one of:
a method for genotyping;
a method for haplotyping; and
a method for analyzing a (genomic) sequence.

In step a) of the method of the invention, a sample comprising the double-stranded nucleic acid molecule is provided and an adapter as described herein is provided, i.e. an adapter wherein one end is closed by a linker. The provided double-stranded nucleic acid molecule preferably comprises a sequence of interest. The sample comprising the double-stranded nucleic acid molecule may be from any source, e.g. of an individual such as a human, animal, plant or microorganism, and the double stranded nucleic acid may be of any kind, e.g. a cellular, cell-free or synthetic double-stranded nucleic acid, for example genomic DNA, chromosomal DNA, artificial chromosomes, plasmid DNA, or episomal DNA, cDNA, RNA, mitochondrial, or of an artificial library such as a BAC or YAC or the like. Preferably, the nucleic acid molecule present in the sample that is used as starting material for the method of the invention is any one of DNA, such as genomic DNA, chromosomal DNA, organellar DNA, nuclear DNA, mitochondrial DNA, artificial chromosomes, plasmid DNA, episomal DNA, cDNA and RNA. Preferably, the DNA is chromosomal DNA, preferably endogenous to the cell. The DNA may also be cDNA reversed transcribed from RNA, wherein said RNA may be from any source, e.g. of an individual such as a human, animal, plant or microorganism. Preferably, the sample is a plant sample.

The nucleic acid molecule is preferably a long nucleic acid molecule, provided e.g. by cell lysis and optionally lysis of an organelle. The nucleic acid molecule for use in the method of the invention may have a size of at least about 50 kb, 100 kb, 150 kb, 200 kb, 300 kb, 400 kb, 500 kb, 600 kb, 700 kb, 800 kb, 900 kb or at least about 1000 kb (1 Mb). The nucleic acid for use in the invention may be a high molecular weight (HMW) nucleic acid or ultra-high molecular weight (uHMW) nucleic acid. HMW nucleic acids may have a length of at least 10 kb. uHMW nucleic acids may have a length of at least 1 Mb. The nucleic acid molecules used in the method of the invention may have a size of at least 1.1 Mb, 1.3 Mb, 1.5 Mb, 1.7 Mb, 2 Mb, 2.5 Mb, 3 Mb, 4 Mb, 5 Mb, 6 Mb, 7 Mb, 8 Mb, 9 Mb or at least about 10 Mb.

Alternatively a nucleic acid molecule, preferably a long nucleic acid molecule, may first be fragmented, resulting in the double-stranded nucleic acid provided in step a). Therefore in an embodiment, step a) of the method provided herein may be preceded by a step of fragmenting a longer nucleic acid molecule. The fragmentation is preferably the fragmentation of a genomic nucleic acid molecule.

The skilled person is familiar with means to fragment nucleic acid molecules and the invention is not limited to any specific means for fragmenting the nucleic acid molecule. The fragmented nucleic acids are preferably fragmented genomic DNA. DNA, and in particular genomic DNA, can be fragmented using any suitable method known in the art. Methods for DNA fragmentation include, but are not limited to, enzymatic digestion and mechanical force.

Non-limited examples of fragmenting the nucleic acid molecule using mechanical force include the use of acoustic shearing, nebulization, sonication, point-sink shearing, needle shearing and French pressure cells.

Preferably, the fragmentation is performed by restriction enzyme and/or site-directed endonuclease digestion. Enzymatic digestion for fragmenting a nucleic acid molecule, includes, but is not limited to, endonuclease restriction. Enzymatic digestion, such as e.g. used in AFLP® and/or Sequenced Based Genotyping technology, may further result in a complexity reduction of the nucleic acid sample. The skilled person knows which enzyme(s) to select for the DNA fragmentation. As a non-limiting example, at least one frequent cutter and at least one rare cutter can be used for the fragmentation of the nucleic acid sample. A frequent cutter preferably has a recognition site of about 3-5 bp, such as, but not limited to MseI. A rare cutter preferably has a recognition site of >5 bp, such as but not limited to EcoRI.

In certain embodiments, in particular when the sample contains or is derived from a relative large genome, it may be preferred to use a third enzyme, rare or frequent cutter, to obtain a larger set of restriction fragments of shorter size.

The step of enzymatic digestion is not limited to any specific restriction endonuclease. The endonuclease may be a type II endonuclease, such as EcoRI, MseI, PstI etc. In certain embodiments a type IIS or type III endonuclease may be used, i.e. an endonuclease of which the recognition sequence is located distant from the restriction site, such as, but not limited to, AceIII, AlwI, AlwXI, Alw261, BbvI, BbvII, BbsI, Bed, Bce831, Bcefl, BcgI, BinI, BsaI, BsgI, BsmAI, BsmFI, BspMI, Earl, Ecil, Eco3II, Eco571, Esp31, FauI, FokI, GsuI, HgaI, HinGUII, Hphl, Ksp6321, MboII, MmeI, MnII, NgoVIII, PleI, RleAI, SapI, SfaNI, TaqJI and ZthII III. Restriction fragments can be blunt-ended or have protruding ends, depending on the endonuclease used.

In a preferred embodiment, the recognition site of at least one of the frequent cutter and the rare cutter is within or in close proximity of the sequence of interest, e.g. the recognition site of the frequent cutter or the rare cutter is located about 0-10000, 10-5000, 50-1000 or about 100-500 bases from the sequence of interest.

In addition or alternatively, the nucleic acid molecule may be digested using a site-directed nuclease, preferably using at least one of a CRISPR nuclease, a zinc finger nuclease, TALENs and meganucleases.

Preferably, the restriction enzyme and/or site-directed endonuclease creates a single-stranded overhang to the double-stranded nucleic acid molecule. The generated single-stranded overhang may provide for adapter ligation or may be part of a primer binding site. Optionally, the ends of the nucleic acid molecule provided in step a) may be repaired, preferably by polishing the ends of the provided nucleic acid molecule. Hence, in case of adapter ligation to the (optionally fragmented) nucleic acid, the method of the invention may comprise a step of polishing the (optionally fragmented) nucleic acid prior thereto. Polishing reactions are well-known in the art and the skilled person straightforwardly understands how to perform polishing reaction, e.g. using a Klenow fragment of DNA polymerase, a T4 DNA polymerase and/or a Mung Bean Nuclease.

In addition or alternatively, the nucleic acid molecule comprising a sequence of interest may be obtained from, or be the result of, amplification of a nucleic acid molecule or fragment.

In addition or alternatively, the nucleic acid molecule provided in step a) may be polished to create blunt ends followed by the addition of a 3′-A staggered overhang, preferably to facilitate ligation to a partly, or fully, double-stranded adapter in step b), wherein said adapter is covalently closed on one end by a linker. Similarly, the addition of a 3′-A overhang, i.e. the addition of a deoxyadenosine nucleotide to the 3′-end of the (polished) nucleic acid provided in a), may be achieved using any conventional method known to the skilled person. The nucleic acid molecule comprising a 3′-A-overhang may subsequently be ligated to a compatible adapter comprising a 3′-T-overhang (preferably the adapter of the invention), having a deoxythymidine nucleotide overhang at its 3′ end. Hence prior to annealing an adapter to the (optionally fragmented) nucleic acid, the method of the invention may optionally comprise a step of A-tailing the, optionally fragmented and/or optionally polished, nucleic acid molecule. A-tailing reactions are well-known in the art and the skilled person straightforwardly understands how to perform an A-tailing reaction, such as e.g. using a Klenow fragment (exo-).

The nucleic acid sample may comprise a plurality of double-stranded nucleic acid molecules. The plurality of nucleic acid molecules may be derived from at least one of the same organism, the same tissue, the same cell, the same organelle and/or the same (fragmented) molecule. Optionally, the sequence of all double-stranded nucleic acid molecules is determined in method provided herein. Alternatively, the sequence of part of the double-stranded nucleic acid molecules is determined. Preferably, (part of) the sequence of a complexity-reduced sample of double-stranded nucleic acid molecules is determined. In an embodiment, the method may comprise a step of enriching the provided sample for the double-stranded nucleic acid molecule of interest, preferably the double-stranded nucleic acid molecule comprising a sequence of interest. Hence, step a) of the method of the invention may be preceded by a step of reducing the complexity of the sample.

In addition or alternatively, the complexity of the sample may be reduced between step b) and c) of the method of the invention. This may be achieved by specifically adding an adapter comprising a linker as defined herein to both ends of a double-stranded nucleic acid comprising a sequence of interest, thereby by closing both ends of the nucleic acid. Optionally, both ends of the nucleic acid are closed by on one end a first adapter comprising the linker as defined herein, and on the other end a second adapter comprising a hairpin structure, wherein preferably the second adapter does not comprise said linker. The closed fragments can subsequently be enriched by degrading the remainder of the nucleic acid molecules or fragments using an exonuclease, as the remainder of the nucleic acid molecules or fragments have at least one open end. The closed fragments can subsequently be opened for instance using a programmable endonuclease, a restriction enzyme recognizing a sequence preferably present in only one of the linked adapters forming a closed end, and/or by mechanical shear stress for instance as induced by a Megaruptor®, rendering two nucleic acids with each having one covalently closed and one open end. For assay designs wherein different adapters are to be ligated to either end of a fragment (for instance, in case one end of a fragment is closed by a linked adapter comprising a restriction enzyme recognition site, and the other end of said fragment is closed by a linked adapter not comprising said restriction enzyme recognition site; or wherein the first adapter comprises an end closed by a linker and the second adapter does not comprise an end closed by a linker), it is noted that the skilled person is aware of means to do so. For instance, a fragment with different ends may be created, e.g. by using different restriction enzymes and/or by adding different nucleotides to a specific end, thereby creating a fragment having one end compatible with only one of the two different adapters, and optionally the other end compatible with only the other one of the two different adapters. Alternatively or in addition, in case the two different adapters are compatible with (and can be ligated to) the same end of the fragment, the adapters may be “mixed” prior to ligating to the combined adapters. As also further indicated herein, admixing of the different adapters in a certain ratio with an amount of fragments prior to ligation, will result in a certain percentage of said fragments comprising both adapters ligated at either end. Thereafter, a site for amplification and/or sequencing may be added to the open end. For instance an adapter for sequencing may be attached to said open end. The adapter may introduce a sequence that enables sequencing of the fragment on an amplification free sequencing platform, wherein preferably the sequencing platform is at least one of nanopore sequencing and PacBio single-molecule sequencing. Preferably, said adapter introduces a leader sequence at the 5′ end of the nucleic acid molecule of the invention, wherein said leader sequence comprises a motor protein for nanopore sequencing.

Addition of an adapter comprising a closed end as defined herein specifically to both ends of a double-stranded nucleic acid comprising a sequence of interest, can be achieved by excising the sequence of interest using one or more (programmed) endonucleases capable of creating an overhang that is compatible to the ligation side of a linker-comprising adapter (i.e. the adapter of the invention).

Another way of selectively sequencing a subset of the nucleic acid molecules and/or fragments in the sample, is by adding in step b) an adapter as defined herein to one end of a double-stranded nucleic acid comprising a sequence of interest, while adding a sequencing primer binding site to the other side of said nucleic acid molecule. Such primer binding sites can be introduced specifically e.g. by prime-editing and/or by specific adapter ligation to compatible overhangs.

The current method as disclosed herein can also be used in Sequence Based Genotyping (SBG) technology, e.g. for polyploid cells. The SBG technology is e.g. described in more detail in WO2007/114693, WO2006/137733 and WO2007/073165, which are incorporated herein by reference. The SBG technology as described in the art can be modified by attaching an adapter comprising a linker as described herein, to the fragmented nucleic acid sample.

In step b) of the method of the invention, the linker-comprising adapter is added to a nucleic acid comprising a sequence of interest. The nucleic acid may be a (partly) double-stranded or a single-stranded nucleic acid. A preferred single-stranded nucleic acid molecule is an RNA molecule or a single-stranded DNA molecule, preferably an RNA or a cDNA molecule, preferably a cDNA molecule. The linker-comprising adapter preferably has a staggered end, preferably a 3′-overhang, such that the adapter end can anneal to nucleotides located at the end, preferably the 3′-end, of the single-stranded nucleic acid comprising a sequence of interest. Hence preferably the linker-comprising adapter is designed such that it can anneal to the 3′-end of the single stranded nucleic acid molecule. Optionally about 70%, 80%, 90%, 95%, 96%, 97%, 98%, 99% or 100% of the nucleotides of the 3′-overhang can anneal to the 3′-end of the single-stranded nucleic acid molecule. Preferably at least the final 1, 2, 3, 4, 5, 6, 7, 8, 9 or final 10 nucleotides of the 3′-overhang can anneal to respectively 1, 2, 3, 4, 5, 6, 7, 8, 9 or 10 nucleotides at the 3′ end of the single-stranded nucleic acid. After annealing the adapter to the single-stranded nucleic acid, the adapter can be ligated to the single-stranded nucleic acid molecule. In addition or alternatively, the single-stranded nucleic acid can be made double-stranded (“filled in” or “elongated”), e.g. using a suitable DNA polymerase or reverse transcriptase. A preferred DNA polymerase is a high fidelity DNA polymerase. Subsequently, the generated double-stranded nucleic acid molecule may be sequenced as described herein, optionally after ligating a second (or further) adapter as described herein.

Preferably, the linker-comprising adapter is added to a (partly) double-stranded nucleic acid molecule. The linker-comprising adapter may be added to at least one end of the provided double-stranded nucleic acid molecule using any conventional means known to the person skilled in the art, such as at least one of adapter ligation, hybridization, annealing and/or tagmentation.

In case both ends of a nucleic acid molecule are closed by an adapter comprising a linker, the molecule is protected against exonuclease degradation as it lacks free “end” nucleotides. For the methods as detailed herein, optionally all nucleic acid molecules that are present in a particular nucleic acid sample are closed at least on one side with a linker, rendering double-stranded nucleic acid molecules that have at least one closed end.

The linker is added by adapter hybridization and/or ligation. In particular, adapters containing a linker on one end of the double-stranded structure can be ligated to the double-stranded molecule. Hence preferably, step b) comprises a step of attaching an adapter to the provided nucleic acid molecule, wherein the adapter is covalently closed on one end by a linker.

The linker adapter is at least partly double-stranded, but may comprise a single-stranded part. Preferably the linker is on one end of the adapter and the other end of the adapter may comprise a single-stranded part. The optionally single-stranded part of the adapter preferably comprises a section, preferably at its 3′ end, that is capable of hybridizing to a nucleic acid molecule provided in step a) of the method described herein. The single-stranded section of the adapter preferably can hybridize to a single-stranded overhang of the nucleic acid molecule, preferably a 3′ overhang of the nucleic acid molecule. The optionally remaining single-stranded part of the annealed adapter may subsequently be filled in, i.e. is made fully double-stranded, using a polymerase, such as, but not limited to, Klenow (known by the skilled person to have 5′→3′ polymerase activity and 3′→5′ exonuclease activity but lacking 5′→3′ exonuclease activity) or a Bst-polymerase (known by the skilled person to be a DNA polymerase from Bacillus stearothermophilus having 5′→3′ polymerase activity and strand displacement activity, but lacking 3′→5′ exonuclease activity). The hybridized adapter may be ligated to the 5′-end of the nucleic acid molecule of the opposite strand to which the adapter is hybridized, prior or after being made double stranded. Alternatively or in addition, the extended 3′-end of the nucleic acid molecule may be ligated to the 5′-end of the polynucleotide that forms the adapter.

The at least partly double-stranded linker-adapter may be ligated to a nucleic acid molecule provided in step a) as defined herein. Preferably, at least 15%, 20%, 25%, 30%, 35%, 40%, 45%, 50%, 55%, 60%, 65%, 70%, 75%, 80%, 85%, 90%, 95% or 100% of the nucleotides in the adapter are double-stranded. The adapter may be 100% or “fully” double-stranded. The adapter may become fully double-stranded after ligation of the adapter to the nucleic acid molecule, e.g. by filing in the single-stranded part of the adapter using a DNA polymerase.

Preferably, one end of the at least partly double-stranded linker-adapter can be ligated to the nucleic acid molecule provided in step a). Hence preferably at least one end of double-stranded end of the adapter can be a blunt or a staggered or “sticky” end. Preferably, the adapter comprises a staggered end. Preferably, the end of the adapter that is ligated to the nucleic acid molecule has an open end that is compatible with an end of said nucleic acid molecule.

The other end of the linker adapter is closed by the linker and cannot be ligated to a nucleic acid molecule or a further adapter. Means for designing and constructing an adapter for use in the method provided herein are well known to the skilled person and the method provided herein is not limited to any particular adapter design and/or construction. The adapter is at least partially double stranded and preferably comprises a closed double-stranded end, wherein the double-stranded end is closed by a linker. The adapter may further have a double stranded structure at or near the side for ligation to the nucleic acid molecule of step (a) of the invention. Preferably, the adapter is substantially fully double-stranded nucleic acid structure, with the exception of the linker and a possible nucleotide-overhang at the ligatable side of the adapter. The ligatable end of the adapter may be blunt or staggered for ligation to a respective compatible blunt or staggered ended nucleic acid molecule. Compatible is to be understood herein as that the overhangs of the adapter and the nucleic acid molecule can hybridize to one another. A nucleic acid fragment produced by restriction enzyme digestion may comprise a 3′ or 5′ overhang, which can hybridize to a complementary (compatible) 3′ or 5′ adapter overhang, respectively.

The at least partly double-stranded adapter can be ligated at its open end to a phosphorylated 5′-end of the nucleic acid molecule. For instance, DNA fragments produced by restriction enzyme fragmentation in general comprise phosphorylated 5′ ends and can therefore readily be ligated. In addition or alternatively, the at least partly double-stranded adapter can be ligated at its open end to a 3′-end of a strand of the nucleic acid molecule, in case the 5′ end of said adapter is phosphorylated. Preferably, the adapter is formed by a single-stranded oligonucleotide comprising a linker, and wherein the oligonucleotide folds into a double-stranded structure comprising a linker at the (closed) end. The 5′ and 3′ end of the oligonucleotide form the open end of the double-stranded structure (i.e. the adapter). The 5′ end of the oligonucleotide is preferably phosphorylated.

The linker may be located exactly in the middle of the modified oligonucleotide forming the adapter of the invention, such that the formed double-stranded structure comprises a blunt open end. Alternatively, the linker is not located exactly in the middle and the resulting double-stranded structure may comprise an open end having a 3′ or a 5′ overhang. Preferably, in case of an overhang adapter, after ligating to a compatible nucleic acid to be sequenced, the position of the linker may be exactly in the middle of the resulting double stranded structure of linked-adapter-ligated nucleic acid. In case one strand of an at least partially double-stranded adapter is ligated, preferably with its 3′ end, to the nucleic acid molecule of step (a) of the invention, the opposite strand of the adapter may be filled in, thus producing a fully double-stranded adapter, preferably a fully double-stranded linked-adapter-ligated nucleic acid. Again, preferably, the linker may be located exactly in the middle of such double-stranded linked-adapter-ligated nucleic acid. Filling in the single-stranded sequence, i.e. to generate a double-stranded sequence can be done using any conventional polymerase, such as, but not limited to Klenow or BST-polymerase. A preferred polymerase is a BST-polymerase.

Optionally, an adapter is ligated to both sides of the nucleic acid molecule, wherein at least one of the adapters comprises an end closed by a linker, in other words, wherein one of the adapter is a linked adapter of the invention. Optionally, the other one of the two adapters does not comprise an end closed by a linker. Said non-linker adapter may have at the non-ligatable end, a single-stranded overhang for e.g. amplification primer binding, sequencing primer binding and/or identification. Optionally, the non-linker adapter may introduce a sequence that enables sequencing of the fragment on an amplification free sequencing platform, wherein preferably the sequencing is on at least one of nanopore sequencing and PacBio single-molecule sequencing platform. Optionally, both strands of said non-linker adapter at the non-ligatable end are single-stranded or non-complementary, thereby forming a Y-shaped adapter, wherein one or both of the strands of the non-complementary end of the adapter may comprise sequences for amplification primer binding, sequencing primer binding, nanopore sequencing and/or identification. Preferably, the 5′-end overhang of the non-ligated side of said non-linker adapter comprises a leader sequence that comprises a motor protein for nanopore sequencing.

Ligation of an adapter can be performed using any conventional method known to the skilled person and the invention is not limited to any specific ligation method or (an effective amount of a) ligation enzyme (ligase). Preferably to facilitate the ligation, the adapter comprises an (open) end that is compatible to at least one end of the nucleic acid molecule. Preferably, the nucleic acid molecule provided in step a) comprises a single-stranded overhang and the adapter provided in step b) comprises an overhang that can be ligated to the overhang of the nucleic acid molecule.

Optionally, a step of fragmentation to provide the nucleic acid of step a) and subsequent adapter ligation of step b) may be combined in a single step, e.g. by means of tagmentation, using a transposase enzyme. When performing a repair step after the transposase reaction most nucleic acid molecules contain adapters at their ends. In this embodiment, the adapter in step b) is ligated by tagmentation, preferably using a Tn5 transposase. Transposases randomly cut the long nucleic acid molecules in shorter nucleic acid molecules and adapters can be ligated on either side of the cleaved points. Tagmentation or “transposase mediated fragmentation and tagging” is a process that is well-known for the person skilled in the art, for example as exemplified in the workflow for Nextera™. The adapters may comprise sequences that make them compatible for use in a tagmentation reaction. Preferably, the adapters used in a tagmentation reaction further comprise a linker at one end. The tagmentation reaction may be followed by a repair step to ensure that all, or substantially all, generated nucleic acid molecules comprise an adapter, preferably on both sides. Hence the nucleic acid molecules comprising a ligated adapter, optionally obtained by tagmentation, may be repaired to remove any single-stranded breaks. Such repair step can be performed using any conventional means known in the art.

An adapter as provided herein, i.e. comprising an end closed by a linker may be added to only one end or to both ends of the provided nucleic acid molecule. As a non-limiting example, a linker may be added to one end of the nucleic acid molecule using a first adapter comprising an end closed by a linker and an optional second adapter of the respective adapter pair that does not comprise an end that is closed by a linker, wherein preferably the second adapter comprises two open ends.

Alternatively, the second adapter may be an adapter comprising an end closed for instance by a linker as defined herein, or a hairpin structure, wherein said second adapter comprises an enzyme recognition site and/or the target sequence for a site-directed nuclease. As further detailed herein, after ligation of the first and second adapter to each of the ends of the provided nucleic acid molecule, the closed fragment can be treated with the respective enzyme in order to cleave the second adapter and obtain a fragment with one open and one closed end.

A first and a second adapter may be combined or “mixed” prior to ligating the combined adapters to the provided nucleic acid molecule, wherein the first adapter comprises an end closed by a linker and the second adapter does not comprise an end closed by a linker. The second adapter preferably comprises two open ends. Optionally, the first and second adapter may only differ by the respective presence and absence of a linker. Alternatively, the second adapter can be a sequencing adapter, preferably an adapter for an amplification free sequencing method. The second adapter can be an adapter for nanopore sequencing or PacBio single-molecule sequencing. The second adapter can be a sequencing adapter, preferably for nanopore sequencing. As a non-limiting example, using an adapter combination wherein about 50% of the adapters are a first adapter as defined herein and about 50% of the adapters are a second adapter as defined herein should result in about 50% of the nucleic acid molecules having an attached first adapter at one end and an attached second adapter at the other end of the same nucleic acid molecule. Step b) may therefore comprise a step b i) of combining (“mixing”) a first and a second adapter as defined herein and a step b ii) of attaching the combined adapters to the provided nucleic acid molecule, wherein preferably the first adapter is attached to one end of the nucleic acid molecule and the second adapter is attached to the other end of the same nucleic acid molecule. Preferably in the adapter combination, there is an excess of the adapter of the invention. Adapter combinations may comprise a first to second adapter (w/w) ratio of about 1:1, or about 2:1, 3:1, 4:1, 5:1, 6:1, 7:1, 8:1, 9:1, 10:1, 11:1, 20:1, 30:1, 40:1, 50:1, 60:1, 70:1, 80:1, 90:1 or about 100:1. Alternatively, the adapter combinations may comprise a first to second adapter (w/w) ratio of about 1:2; 1:3; 1:4, 1:5, 1:6, 1:7, 1:8, 1:9, 1:10, 1:11 or about 1:12.

Alternatively or in addition, the adapter comprising the linker may be capable of ligation to only one end of the provided nucleic acid molecule. As a non-limiting example, the adapter may comprise an end, preferably a 3′-end, that is capable of hybridizing to only one end of the provided nucleic acid molecule. E.g. if the nucleic acid molecule comprises dissimilar ends, such as a blunt end and a sticky end, the ligatable end of the adapter may comprise a respectively a blunt or a (compatible) sticky end, such that it can be ligated to only one end of the nucleic acid molecule. Equally the nucleic acid molecule may comprise two different overhangs and the adapter may be compatible with only one of these overhangs. Consequently, the adapter may be ligated to only one end of the provided nucleic acid molecule. Optionally, an adapter comprising a linker at one end may be added to both ends of the provided nucleic acid molecule.

The adapter(s) for use in a method as defined herein preferably do(es) not comprise a recognition site for the restriction endonuclease or the site-directed endonuclease that can be used in step b3) of the method provided herein.

In step b) of the method of the invention, a double-stranded nucleic acid molecule is generated having one open end and one closed end, comprising adding an adapter to an end of the double-stranded nucleic acid molecule provided in step a), wherein the adapter has been closed on one end by a linker. In other words, the nucleic acid molecule provided in step a) is modified to generate a double-stranded nucleic acid molecule having one open end and one closed end. By said linker addition at least one end of the double-stranded nucleic acid molecule is closed to provide a double-stranded nucleic acid molecule having one open end and one closed end. A terminus of a double-stranded nucleic acid, wherein the 3′-end terminal nucleotide of the respective upper strand is covalently linked to the 5′-end terminal nucleotide of the respective bottom strand, is annotated herein as a “closed end”. Likewise, a terminus of a double-stranded nucleic acid, wherein the 5′-end terminal nucleotide of the respective upper strand is covalently linked to the 3′-end terminal nucleotide of the respective bottom strand, is also annotated herein as a “closed end”. A “closed end” is thus understood herein as a terminus of a double-stranded nucleic acid wherein said terminal nucleic acids from opposite strands are covalently linked to each other, as opposed to an “open end” which is understood herein as a terminus of a double-stranded nucleic acid wherein said terminal nucleic acids from opposite strands are not covalently linked to each other.

The end of the double-stranded nucleic acid is closed by a linker, preferably a linker as described herein. Hence preferably the double-stranded nucleic acid is closed using a non-naturally occurring chemical moiety, i.e. preferably the double-stranded nucleic acid is not closed by a hairpin consisting of (naturally occurring) nucleotides. Preferably, the at least one closed end of the double-stranded nucleic acid is obtained by ligating an adapter as described herein to the nucleic acid molecule.

In case the adapter is ligated to only one end of the nucleic acid molecule provided in step a), the resulting double-stranded nucleic acid molecule will have one open and one closed end.

Optionally, the adapter as described herein can be ligated to both ends of the provided nucleic acid molecule. In this embodiment, the resulting double-stranded nucleic acid molecule will thus have two closed ends. Step b) may therefore comprise the following (sub-) steps of:
a step b1) of ligating an adapter as described herein, i.e. an adapter having one end closed by a linker, to both ends of the double-stranded nucleic acid molecule. The resulting double-stranded nucleic acid molecule will thus have both ends closed by a linker.
An optional step b2) of contacting the nucleic acid sample with an exonuclease. The exonuclease can digest any remaining nucleic acid molecule comprising at least one open end, thereby enriching the sample for the nucleic acid molecule comprising closed ends on both sides.
A step b3) of cleaving the double-stranded nucleic acid molecule having two closed ends, thereby generating a double-stranded nucleic acid molecule having one open end and one closed end.
   The method may comprise an optional step b2) of contacting the nucleic acid molecule with an (effective amount of an) exonuclease. The exonuclease may digest any nucleic acid molecule not comprising two closed ends, i.e. comprising one or two open ends. Such nucleic acid molecules are for example, but not limited to, nucleic acid molecules without adapters, nucleic acid molecules with one or two adapters having an open end, and/or cleaved nucleic acid molecules having one open end and one closed end.

The sample provided in step a) may thus comprise a plurality of nucleic acid molecules and in step b1) part of the nucleic acid molecules are covalently closed by adapter ligation, i.e. part of the nucleic acid molecules comprise two closed ends. Optional step b2) is thus a step of the removing open-ended double-stranded nucleic acid molecules by exposing the sample to an exonuclease. The nucleic acid molecules having two closed ends are protected from degradation, while the non-protected fragments are degraded, resulting in enrichment or complexity reduction of the nucleic acid molecules comprising the sequence of interest. Therefore in an embodiment, the method of the invention takes the approach of removal of an undesired (non-target) part of the nucleic acid sample. As a non-limiting example, the adapters in step b) may be ligated to nucleic acid molecules having a selective staggered overhang, for example created by enzymatic digestion. The molecules comprising the adapters are thus closed on both ends, and the exonuclease treatment in step b2) may digest any nucleic acid molecule not having two closed ends. The exonuclease treatment in step b2) may thus result in an enrichment of nucleic acid molecules comprising closed ends.

The exonuclease may be an exonuclease I, III, V, VII, VIII, or related enzyme, or any combination thereof. Exonuclease Ill recognizes nicks and extend the nick to a gap until a piece of ssDNA is formed. Exonuclease VII can degrade this ssDNA. Exonuclease I also degrades ssDNA. ExoIII and ExoVII is a preferred combination of exonucleases for use in step b2) of the method described herein.

Exonuclease V is capable of degrading ssDNA and dsDNA in both 3′ to 5′ and in 5′ to 3′ direction. Therefore in a preferred embodiment, the exonuclease in step b2) of the method described herein is an exonuclease that is capable of degrading ssDNA and dsDNA in both 3′ to 5′ and in 5′ to 3′ direction, preferably an exonuclease V.

Further information on methods for degrading non-target sequences is provided in U.S. Patent Publication No. 2014/0134610, which is incorporated herein by reference in its entirety for all purposes.

Step b2) is preferably performed at conditions (e.g. time, temperature, reaction buffer, enzyme concentration, etc) sufficient for the exonuclease to degrade substantially all non-protected nucleic acid molecules. Preferably, step b2) is performed at conditions and time sufficient for the exonuclease to degrade all non-protected nucleic acid molecules. Step b2) is preferably performed for about 1 minute to about 12 hours, preferably 30 min, at about 20-80° C., preferably about 37° C.,

After exonuclease treatment in step b2), the exonuclease may be inactivated by, for example, but not limited to, at least one of a Proteinase, e.g. Proteinase K, treatment or heat inactivation. Such techniques are standard in the art and the skilled person straightforwardly understands how to inactivate an exonuclease. A preferred inactivation step is heating the sample at a temperature of about 50-90° C., preferably about 75° C., for about 1-120 minutes, preferably about 10 minutes.

Optionally, both ends of a double-stranded nucleic acid molecule is closed. The molecules that are closed on both ends are insensitive for 5′ or 3′ modifying enzymes. As indicated above, an optional step of exonuclease treatment can be added to remove any possible nucleic acid molecules that are not covalently closed on both ends. Subsequently, the (covalently closed) nucleic acid molecules can be selectively opened by using for instance (an effective amount of) a restriction endonuclease or site-directed endonucleases. Although all nucleic acid molecules are still present in the reaction mixture, only those cleaved in the last opening reaction are able to be used in a subsequent (sequencing) process, for instance by ligating sequencing adapters to the opened ends thereby selectively rendering these opened fragments ready for sequencing. Alternatively, the opened fragments may be degraded using exonuclease treatment, thereby enriching for the non-opened nucleic acid molecules for further processing. For instance, these non-opened molecules may be opened in a second round of selective opening using for instance site-directed endonucleases targeted to these non-opened molecules.

The method described herein may further comprise a step b3) of cleaving the closed double-stranded nucleic acid molecule, thereby generating a double-stranded nucleic acid molecule having one open end and one closed end. “Cleaving” is understood herein the generation of a double-stranded break. The double-stranded break may be created by the use of a (n) (endo) nuclease or by the use of two nickases that cleave opposite stands. The double-stranded break may create a blunt open end of the nucleic acid molecule. After cleavage the cleaved nucleic acid molecule may thus have one open blunt end and one closed end. Alternatively, the double-stranded break may create a staggered open end of the cleaved nucleic acid molecule. After cleavage, the cleaved nucleic acid molecule may thus have one open staggered end and one closed end. The double-stranded nucleic acid molecule comprising two closed ends may be cleaved at a target sequence located in one of the attached adapters, and/or may be cleaved at a target sequence located in the double-stranded nucleic acid molecule.

In an embodiment, an adapter, optionally a further adapter, for use in the method provided herein further comprises a restriction enzyme recognition site or target sequence for a site-directed nuclease between the linker (or hairpin) and the part of the adapter for ligation to the nucleic acid molecule. Preferably, such adapter is located at only one side of the nucleic acid molecule of the invention, while an adapter located at the opposite side of said molecule comprises a linker but lacks said restriction enzyme recognition or target site. After step b1) of closing both ends of the nucleic acid molecule, the nucleic acid molecule may be contacted with a restriction enzyme or site-directed nuclease, resulting in a double stranded nucleic acid molecule having one closed and one open end.

One end of the nucleic acid molecule comprising two closed ends may be opened by fragmentation and/or tagmentation. Preferably, the nucleic acid molecule in step b3) is cleaved by a site-directed endonuclease or a restriction endonuclease. Optionally, all nucleic acid molecules comprising two closed ends will be cleaved in step b3) by the endonuclease and subsequently sequenced in step c) of the method provided herein. Optionally only a part or “subset” of the nucleic acid molecules comprise a target sequence that is recognized by the endonuclease. Hence optionally only a part of the nucleic acid molecules comprising two closed ends will be cleaved by the endonuclease and subsequently sequenced in step c) of the method provided herein. The selective opening of the nucleic acid molecule thus results in an enrichment of the nucleic acid molecules comprising a sequence of interest. Preferably, the closed nucleic acid molecule comprises a single sequence that is targeted by the endonuclease. Optionally, the nucleic acid molecule may comprise the target sequence more than once, e.g. the nucleic acid molecule may comprise the target sequence 1, 2, 3, 4, 5, 6 or more times.

The nucleic acid molecule comprising closed ends may be cleaved by a restriction endonuclease. Any sequence-specific endonuclease may be suitable for use in the method provided herein. The endonuclease may be a so-called “restriction endonuclease” or “restriction enzyme”, e.g. a Type I, Type II, Type III, Type IV or Type V restriction endonuclease. A preferred restriction endonuclease is a Type II restriction endonuclease, preferably Type IIP or Type IIS. In case a fragmentation in step a) is performed by cleaving the DNA with a restriction enzyme, the enzyme used in step b3) is preferably a different restriction endonuclease.

The nucleic acid molecule may be cleaved by a site-directed nuclease. A site-directed nuclease may be selected from the group consisting of a TALENs, an RNA-guided CRISPR nuclease, a zinc finger nuclease and a meganuclease. Preferably, the site-directed nuclease is a TALENs or an RNA-guided CRISPR (Clustered Regularly Interspaced Short Palindromic Repeats) nuclease.

Preferably, the CRISPR nuclease is a Type II CRISPR-nuclease, e.g., Cas9 (e.g., the protein of SEQ ID NO: 1, encoded by SEQ ID NO: 2, or the protein of SEQ ID NO: 3) or a Type V CRISPR-nuclease, e.g. Cpf1 (e.g., the protein of SEQ ID NO: 4, encoded by SEQ ID NO: 5) or Mad7 (e.g. the protein of SEQ ID NO: 6 or 7), or a protein derived thereof, having preferably at least about 70%, 80%, 90%, 91%, 92%, 93%, 94%, 95%, 96%, 97%, 98%, or 99% sequence identity to said protein over its whole length. Preferably, the site-directed nuclease is a Type II CRISPR-nuclease, preferably a Cas9 nuclease. The skilled person knows how to obtain the site-directed nuclease for use in the method of the invention, such as a TALEN or a CRISPR-nuclease.

In the prior art, numerous reports are available on the design of a CRISPR-nuclease and its use. See for example the review by Haeussler et al (J Genet Genomics. (2016) 43 (5): 239-50. Doi: 10.1016/j.jgg.2016.04.008.) on the design of guide RNA and its combined use with a CAS-protein (originally obtained from S. pyogenes), or the review by Lee et al. (Plant Biotechnology Journal (2016) 14 (2) 448-462). Optionally, the site-directed nuclease is a CRISPR-nuclease being either a nickase or (endo) nuclease.

The site-directed nuclease for use in the method of the invention may comprise or consist of a whole type II or type V CRISPR-nuclease or variant or functional fragment thereof. Preferably, the site-directed nuclease is a Cas9 protein. The Cas9 protein may be derived from the bacteria Streptococcus pyogenes (SpCas9; NCBI Reference Sequence NC_017053.1; UniProtKB-Q99ZW2), Geobacillus thermodenitrificans (UniProtKB-A0A178TEJ9), Corynebacterium ulcerous (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis/(NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1). Encompassed are Cas9 variants from these, having an inactivated HNH or RuvC domain homologues to SpCas9, e.g. the SpCas9_D10A or SpCas9_H840A, or a Cas9 having equivalent substitutions at positions corresponding to D10 or H840 in the SpCas9 protein, rendering a nickase.

The site-directed nuclease may be, or may be derived from, Cpf1, e.g. Cpf1 from Acidaminococcus sp; UniProtKB-U2UMQ6. The variant may be a Cpf1-nickase having an inactivated RuvC or NUC domain, wherein the RuvC or NUC domain has no nuclease activity anymore. The skilled person is well aware of techniques available in the art such as site-directed mutagenesis, PCR-mediated mutagenesis, and total gene synthesis that allow for inactivated nucleases such as inactivated RuvC or NUC domains. An example of a Cpf1 nickase with an inactive NUC domain is Cpf1 R1226A (see Gao et al. Cell Research (2016) 26:901-913, Yamano et al. Cell (2016) 165 (4): 949-962). In this variant, there is an arginine to alanine (R1226A) conversion in the NUC-domain, which inactivates the NUC-domain.

The site-directed nuclease may be, or may be derived from, CRISPR-Caso, a nuclease that is about half the size of Cas9. CRISPR-Caso uses a single crRNA for targeting and cleaving the nucleic acid as is described e.g. in Pausch et al (CRISPR-CasΦ from huge phages is a hypercompact genome editor, Science (2020); 369 (6501): 333-337).

An active, partly inactive or a dead site-directed nuclease, preferably an active, partly inactive or a dead CRISPR-nuclease complex may serve to guide a fused functional domain to a specific site in the nucleic acid molecule as determined by the guide RNA. Hence, the site-directed nuclease may be fused to a functional domain. Preferably, such functional domain is an endonuclease domain. In an embodiment, an inactive CAS protein for use in a method as defined herein (e.g. dCas9, dCpf1) is fused to a restriction enzyme such as, but not limited to, Fok1 or Clo51, preferably as described in WO2014/144288, WO2016/205554, Tsai et al. Nat Biotechnol. 2014 June; 32 (6): 569-576, or Cheng et al Biotechnol J. 2022 July; 17 (7): e2100571, all of which are incorporated herein by reference). Preferably, the fusion protein has a sequence of any one of SEQ ID NO: 8-10, or a sequence having at least about 50%, 55%, 60%, 65%, 70%, 75%, 805, 85%, 90%, 95%, 96%, 97%, 98%, 99% or 100% sequence identity with any one of SEQ ID NO: 8-10.

The nucleic acid molecule comprising two closed ends may be cleaved with a guide RNA-CAS complex. The guide RNA directs the complex to a defined target site in a double-stranded nucleic acid molecule, also named the protospacer sequence. The guide RNA comprises a sequence for targeting the site-directed nuclease complex to a protospacer sequence that is preferably near, at or within a sequence of interest in the double-stranded nucleic acid molecule.

The guide RNA may be a guide RNA that is a single guide (sg) RNA molecule, or the combination of a crRNA and a tracrRNA (e.g. for Cas9) as separate molecules, or a crRNA molecule only (e.g. in case of Cpf1 and Caso). Optionally, the guide RNA is a single guide (sg) RNA (e.g. for Cas9) or a crRNA only (e.g. in case of Cpf1 and Caso).

The guide RNA for use in a method provided herein may comprise a sequence that can hybridize to a sequence in the double-stranded nucleic acid molecule, preferably to or near a sequence of interest, preferably a sequence of interest as defined herein. The guide RNA preferably comprises a nucleotide sequence that is fully complementary to a sequence located in the double-stranded nucleic acid molecule, optionally fully complementary to a sequence located in the sequence of interest i.e. the sequence of interest may comprise a protospacer sequence.

Alternatively or in addition, the nucleic acid molecule comprising two closed ends can be cleaved with a site-directed endonuclease, wherein the site-directed endonuclease is a TALENs. TALENs are well-known for the person skilled in the art and are constructed by fusing a TAL effector DNA binding domain (TALE) to an effector domain, preferably a (non-specific) DNA cleavage domain, such as a FokI cleavage domain. The term “Transcriptional Activator-Like Effector”, “TALE” or “TAL effector DNA binding domain” as used herein, refers to proteins comprising a DNA binding domain, which contains a highly conserved 33-34 amino acid sequence comprising a highly variable two-amino acid motif (Repeat Variable Di-residue, RVD). The RVD motif is known to determine the binding specificity to a nucleic acid sequence, and can be engineered according to methods well known to those of skill in the art to specifically bind a desired DNA sequence (see, e.g., WO2010/079430, WO2011/072246 and WO2015027134, the entire contents of each of which are incorporated herein by reference). The simple relationship between amino acid sequence and DNA recognition has allowed for the engineering of specific DNA binding domains by selecting a combination of repeat segments containing the appropriate RVDs.

The term “Transcriptional Activator-Like Element Nuclease” or “TALEN” as used herein, refers to an engineered nuclease, comprising a transcriptional activator like effector DNA binding domain (TALE) linked to a DNA cleavage domain, for example, a FokI domain. A number of modular assembly schemes for generating engineered TALE constructs have been reported previously and are known for the person skilled in the art.

Transcription activator-like effector nucleases (TALENs) are fusions of a restriction endonuclease cleavage domain, preferably a FokI domain, with a DNA-binding transcription activator-like effector (TALE) repeat array. Other useful endonuclease domains may include, for example, HhaI, HindIII, NotI, BbvCI, EcoRI, BgI II and AlwI. Preferably, the cleavage domain is a FokI domain.

The method as detailed herein further comprises a step c) of sequencing both strands of at least part of the double-stranded nucleic acid molecule in a single sequencing reaction to generate a duplex read. The double-stranded nucleic acid obtained after step b) of the method provided herein comprises one open end and one closed end. Using a single sequencing reaction, both strands are sequenced directly after each other, generating a duplex read.

Prior to sequencing a further adapter can be attached to the nucleic acid molecule. The further adapter is preferably attached to the open end of the double-stranded nucleic acid molecule. The further adapter may be an adapter suitable for amplification and/or sequencing, preferably, the further adapter is a sequencing adapter. Hence preferably, a sequencing adapter is ligated to the open end of the double-stranded nucleic acid after step b) and prior to step c). The further adapter may be a sequencing adapter comprising a functional domain that allows for deep-sequencing. Preferably, the further adapter may introduce a sequence that allows for sequencing on an amplification free sequencing platform, wherein preferably the sequencing platform is at least one of nanopore sequencing and PacBio single-molecule sequencing. The further adapter may be a sequencing adapter comprising a functional domain that allows for nanopore sequencing such as Oxford Nanopore Technologies (ONT), Ontera sequencing or Complete Genomics sequencing. Preferably, the further adapter allows for Oxford Nanopore Technologies (ONT) sequencing, preferably a nanopore selective sequencing method.

Hence preferably, the further adapter comprises at least one sequencing primer binding site and/or the further adapter comprises at least one amplification primer binding site. The further adapter may comprise at least two sequencing primer binding sites and/or the further adapter may comprise at least two amplification primer binding site. The additional adapter may be a single-stranded, double-stranded, partly double-stranded, Y-shaped or a hairpin nucleic acid molecule. Preferably, the further adapter is a (partly) double-stranded adapter comprising two open ends. Optionally the further adapter comprises an identifier sequence, preferably an identifier sequence as defined herein.

After step b) and prior to sequencing at least part of both strands of the nucleic acid molecule, and preferably prior to attaching the optional further adapter, the double-stranded nucleic acid molecule comprising one open and one closed end may be repaired, preferably by polishing the ends of the nucleic acid molecule. Hence prior to sequencing and prior to attaching the optional further adapter, the method as provided herein may optionally comprise a step of polishing the open end of the nucleic acid molecule.

In addition or alternatively, the nucleic acid molecule comprising one open and one closed end may be modified to comprise an A-overhang, preferably to facilitate ligation to the further adapter, wherein the further adapter preferably comprises a T-overhang. Hence prior to annealing the further adapter to the open end of the nucleic acid molecule, the method of the invention may optionally comprise a step of A-tailing the, optionally polished, nucleic acid molecule.

In step c) of the method of the invention, the double-stranded nucleic acid molecule comprising one open end and one closed end is sequenced to generate a duplex read. Part of the nucleic acid molecule may be sequenced, wherein the sequenced part comprises part of the forward strand and part of the complementary reverse strand.

A duplex read is a sequencing read comprising a sequence of the forward strand of a double-stranded nucleic acid molecule, followed by the sequence of the reverse strand of the same double-stranded nucleic acid molecule. The double-stranded nucleic acid molecule comprising one open and one closed end is denatured, such that the forward and reverse strand form a single-stranded template, connected by the closed end of the nucleic acid molecule (see FIG. 1). The linker forming the closed end are thus located in between the sequence of the forward strand and the sequence of the reverse strand. Preferably, the linker forming the closed end are in the middle of the single-stranded template.

The linker preferably results in a signal that can be detected during sequencing. The detection of such signals have been described in the art for e.g. amplification free sequencing platforms. For example, Tse et a (Proc Natl Acad Sci USA. 2021; 118 (5): 1-11) disclose the use of the PacBio platform to detect modified cytosines within a DNA strand. Moreover, nanopore sequencing produces a raw output of current intensity (picoamps) over time (milliseconds). This output signal is often referred to as “the squiggle”. The linker may result in a modified signal produced during real-time sequencing or in the absence of a signal for a predetermined period of time (see FIG. 1, where this is exemplified by a straight line in a squiggle signal), indicated herein as a “signature sequence lag”. Preferably the signature sequence lag is minimal or absent. Preferably, the position of the linker is known. In addition or alternatively, the position of the linker may be marked using a signatory sequence present in adapter elements flanking the linker element. It can therefore be accurately assessed at what position in the read the reading of the forward strand ends and the reading of the reverse strand begins. In other words, the position of the linker can be used to quickly identify the sequence of interest and its complementary sequence within the nucleic acid molecule to be sequenced. Hence, the use of a linker may significantly increase the accuracy of duplex read calling.

The single-stranded nucleic acid molecule or “opened double-stranded nucleic acid molecule” is sequenced in a single sequencing reaction. A generated duplex read thus preferably comprises the following successive sequences: a first sequence read comprising (part of) the forward strand and an optional signatory sequence at the end of the first sequence read, a (minimal or absent) signatory sequence lag, and a second sequence read comprising (part of) the sequence of the reverse strand read optionally comprising the reverse of the signatory sequence at the beginning of the second sequence read.

Preferably at least about 5%, 10%, 20%, 30%, 40%, 50%, 60%, 70%, 80%, 90% or at least 95% of the nucleic acid molecule is sequenced. Preferably, the complete nucleic molecule may be sequenced. Hence preferably the complete forward strand of the nucleic acid molecule (providing for the first sequence block of the sequence read), and the complete reverse strand of the nucleic acid molecule (providing for the second sequence block of the sequence read) is sequenced in a single sequencing reaction.

Preferably, the prepared nucleic acid molecule is deep-sequenced. Preferably, the sequencing of step c) of the method provided herein is an amplification free sequencing method. The sequencing is preferably at least one of nanopore sequencing and PacBio single-molecule sequencing. Preferably, the sequencing of step c) of the method provided herein is nanopore sequencing. Nanopore sequencing technologies include but are not limited to Oxford Nanopore sequencing technologies (e.g., GridION, MinION) (see for example Logsdon et al., Nat. Rev. Genet. 2020; 21 (10): 597-614).

The prepared nucleic acid molecule can be sequenced by nanopore selective (“Read Until”) sequencing. In nanopore selective sequencing, during real time sequencing the generated data (either direct current signals or base calls translated from these current signals) is compared to one or more reference sequence(s). In case a set number of nucleotides or amount of signals of the target sequence align with the reference sequence, sequencing will proceed, if not, the current is reversed thereby removing the nucleic acid from the pore and making the pore available for sequencing of a new nucleic acid. The set number of nucleotides may be at least the first 50, 60, 70, 80, 90, 100, 150, 200, 250, 300, 350, 400, 450, or 500 nucleotides of the nucleic acid read. The one or more reference sequences may be a multitude of different sequences. Preferably, each of these reference sequences is at least 50, 60, 70, 80, 90, 92, 93, 94, 95, 96, 97 98, 99 or 100% identical to the sequence of the nucleic acid molecule obtained by the method of the invention. In an embodiment, each of the reference sequences is at least 50, 60, 70, 80, 90, 92, 93, 94, 95, 96, 97 98, 99 or 100% identical to the sequence of a particular subset of nucleic acid molecules obtained by the method of the invention. One of the benefits of selectively sequencing a particular subset by nanopore selective sequencing is that in different sequencing runs, different subsets may be sequenced using the prepared library of nucleic acid molecules having one open end and one closed end.

The sequence read lengths obtained by long-read sequencing in the method of the invention can be at least about 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 200 kb, 300 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, or 10 Mb.

The method provided herein further comprises a step d) of generating a consensus sequence from the duplex read. The duplex read comprises the sequence of the forward strand, an optional sequence lag, and the sequence of the reverse strand of the same double-stranded nucleic acid molecule. The obtained sequence of the forward strand and the reverse strand can be combined or “collapsed” into a consensus sequence. Optionally, the adapter provided herein comprises a signatory sequence (or a barcoding nucleotide sequence) that can be used to filter for genuine duplex reads and/or mark the end of the forward read and start of the reverse read prior to collapsing the read into a consensus sequence.

Also provided is a bio-informatics analysis protocol, and a computer medium instructed to perform said bio-informatics analysis protocol, making use of prior knowledge of the position of the linker and/or the signature sequence for analyzing duplex reads and thereby accurately determining the sequence of interest. Optionally, normal duplex analysis or split duplex analysis protocols can be used for the analysis of the sequence of interest, preferably modified to detect the signatory sequence within the double-stranded nucleic acid molecule having one open end and one closed end obtainable by step b) of the method of the invention.

A normal duplex analysis protocol detects two separate sequences (i.e. the forward strand and the reverse strand) and subsequently combines these two sequences into a consensus sequence. The split sequencing protocol detects a single sequence, comprising the forward strand and the reverse strand. The forward strand and reverse strand are subsequently split and combined into a consensus sequence. Optionally, the bio-informatics analysis protocol, preferably the split sequencing protocol, can be adapted to the type of linker used for closing the double-stranded molecule. Optionally, the bio-informatics analysis protocol, preferably the split sequencing protocol, can be adapted to the type of signature sequence used to mark the position of the linker within the nucleic acid to be sequenced. Put differently, the protocol can be trained where to split the sequence into a forward and reverse strand to generate a consensus sequence.

Hence the method of the invention improves the sequencing accuracy, preferably to a modal accuracy of about Q30. An accuracy of Q30 is equivalent to the probability of an incorrect base call 1 in 1000 times, i.e. the base call accuracy is 99.9%. The consensus sequence can optionally be aligned or compared to a known nucleotide sequence, such as, but not limited to, a known genomic sequence. As a non-limiting example, if the same nucleotide mutation is detected in both the forward and the reverse strand, there is a significantly increased likelihood that the mutation is thus a genuine mutation and not e.g. a sequencing artifact. Generating a consensus sequence from the duplex read thus results in the determination the sequence in the double-stranded nucleic acid molecule, preferably with an high accuracy, preferably with a modal accuracy of about Q30.

Preferably, the method is performed for a plurality of samples. Optionally, the method of the invention is multiplexed, i.e. applied simultaneously for multiple nucleic acid samples, such as for at least about 10, 20, 30, 40, 50, 60, 70, 80, 90, 100, 500, 1000 or more nucleic acid samples. The method may thus be performed in parallel on a plurality of samples, wherein “in parallel” is to be understood herein as substantially simultaneously but each sample being processed in a separate reaction tube or vessel.

In addition or alternatively, one or more steps of the method provided herein may be performed on a plurality of pooled samples. The plurality of samples are preferably pooled prior to step c). Optionally, the plurality of samples are pooled prior to step b). In order to trace back the double-stranded nucleic acid molecules to the originating sample, the nucleic acid molecules may be tagged with an identifier prior to pooling the samples. Such an identifier can be any detectable entity, such as, but not limited to, a radioactive or fluorescent label, but preferably is a particular nucleotide sequence or combination of nucleotide sequences, preferably of defined length. In addition or alternatively, the samples can be pooled using a clever pooling strategy, such as, but not limited to, a 2D and 3D pooling strategy, such that after pooling each sample is encompassed in at least two or three pools, respectively. A particular nucleic acid molecule can be traced back to the originating sample by using the coordinates of the respective pools comprising the double-stranded nucleic acid molecules. The plurality of samples may be pooled prior to step c) and/or prior to step b).

In between one or more of the steps of the method as provided herein, the nucleic sample may be purified and/or the reaction enzyme may be inactivated. A purification step, e.g., an AMPure bead-based purification process, may be included to remove complexes, enzymes, free nucleotides, possible free adapters, and possible small, non-relevant, nucleic acid molecules. The nucleic acid molecule may be recovered after purification and subjected to further processing and/or analysis, such as single-molecule sequencing.

An optional purification step is a proteinase K treatment. Alternatively or in addition, said purification may comprise the following steps:
I. exposing the nucleic acid sample to one or more solid supports that specifically and effectively bind the nucleic acid molecule; and optionally,
II. washing the one or more solid supports and eluting nucleic acid molecule from the one or more solid supports.

The one or more solid supports may be, but not limited to, AMPure beads. After purification, at least one purified nucleic acid molecule is obtained.

The method of the invention may further comprise a size-selection step, for example to remove any non-ligated adapters or adapter-adapter pairs. Optionally, the size-selection step is performed prior to step c) of the method of the invention. Alternatively, there is no further purification, inactivation and/or size selection step.

In an embodiment, the method provided herein is a sequencing method that is free of amplification and/or cloning steps. Reduction of amplification steps is beneficial, as epigenetic information (e.g., 5-mC, 6-mA, etc.) will get lost in amplicons. Further amplification can introduce variations in the amplicons (e.g., via errors during amplification) such that their nucleotide sequence is not reflective of the original sample.

In an aspect, provided herein is a kit of parts for performing the method described herein. Preferably, the kit of parts is for use in a method as defined herein. Preferably, the kit of parts comprises at least one or more adapters as defined herein, comprising an open end and a closed end, wherein the end is closed by a linker. A plurality of adapters may be combined in one vial or may be present in separate vials, e.g, wherein the adapters of one vial comprise the same identifier sequence, preferably the same sample identifier sequence. Preferably wherein the adapters present in separate vials comprise different identifier sequences, preferably different sample identifier sequences.

Alternatively or in addition, the kit of parts comprises at least one or more sequencing adapters. A plurality of sequencing adapters may be combined in one vial or may be present in separate vials, e.g, wherein the sequencing adapters of one vial comprise the same identifier sequence, preferably the same sample identifier sequence. Preferably wherein the sequencing adapters present in separate vials comprise different identifier sequences, preferably different sample identifier sequences. The kit of parts may comprise one or more reagents for performing an ONT sequencing reaction. Hence the kit of parts may comprise at least one of:
one or more vials comprising adapters comprising an open end and a closed end, wherein the closed end is closed by a linker; and
one or more vials comprising a further adapter as defined herein for sequencing, preferably for an ONT sequencing reaction.

Preferably, the kit of parts may further comprise at least one of:
one or more vials comprising a restriction endonuclease;
one or more vials comprising at a gRNA-CAS complex as defined herein, or one or more constructs encoding the same;
one or more vials comprising a gRNA for complexing with a CRISPR-CAS protein to form a gRNA-CAS complex, and a further vial comprising said CRISPR-CAS protein or a construct encoding the same; and
a further vial comprising one or more exonucleases.
   Preferably, the kit comprises at least 2, 4, 10, 20, 30, or 50 vials comprising one or more gRNAs as defined herein. Preferably, the volume of any of the vials within the kit do not exceed 100 mL, 50 mL, 20 mL, 10 mL, 5 mL, 4 mL, 3 mL, 2 mL or 1 mL.

The reagents may be present in lyophilized form, or in an appropriate buffer. The kit may also contain any other component necessary for carrying out the present invention, such as buffers, pipettes, microtiter plates and written instructions. Such other components for the kits of the invention are known to the skilled person.

Having now generally described the invention, the same will be more readily understood through reference to the following example which is provided by way of illustration and is not intended to be limiting of the present invention.

### FIGURE LEGEND

FIG. 1: Schematic overview of library preparation of λ DNA HindIII digest. In step a, index adapters (hatched) are ligated to fragments of λ DNA HindIII digest; depicted is a PvuI restriction site-comprising fragment, wherein each strand is indicated as a black bar and the restriction site as a white bar). After DNA repair and A-addition, indexed fragments are closed by ligation of linked adapters (spotted, with linker) in step b. In step c, non-closed fragments are removed by exonuclease treatment. Subsequently, closed fragments are opened by PvuI restriction (step d), adapters for nanopore sequencing are added (white bars at the open ends of the fragment, with helicase motor proteins in white circles) (step e) and fragments are sequenced (step f).

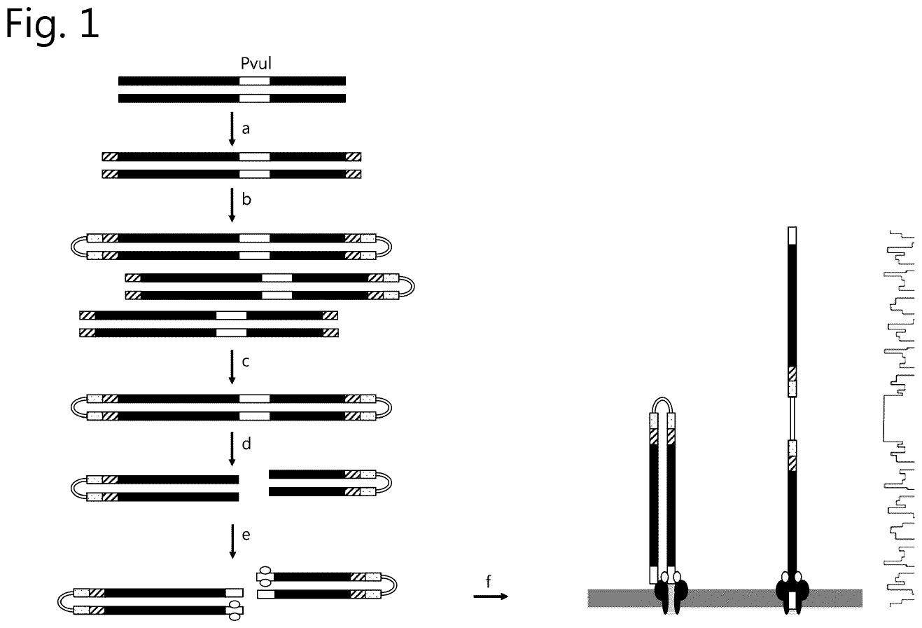

FIG. 2: Schematic overview of sgRNA-guided (red arrow heads) CRISPR-Cas9 restriction of A DNA. Three CRISPR-Cas9 generated DNA fragments (17.5 Kb, 15 Kb and 12.5 Kb) each comprise one recognition site for restriction enzyme BSaI or XbaI (blue arrow heads). Fragment lengths after BSaI or XbaI digested are indicated with broken lines and Cos-end fragments indicated in dotted lines.

FIG. 3: Tapestation profiles show successful generation of DNA fragments of expected lengths after CRISPR-Cas9 digestion, (A) gel based visualisation and (B) electropherogram report is shown.

### EXAMPLES

### Example 1

A sequencing library using the adapters of the invention was used to show that the method as detailed herein indeed increases the accuracy of nanopore deep-sequencing. A λ DNA-Hindill digest (New England Biolabs; N3012; 500 ug/ml) was prepared as template DNA.

In short, adapters having a covalently closed end and an open end were added for ligation to both ends of the λ DNA-HindIII digested nucleic acid fragments, resulting in covalently closed nucleic acid fragments. By ExoV treatment any non-closed nucleic acids were removed. Subsequent PvuI digestion resulted in opening of one end of the covalently closed λ DNA-HindIII-digested fragments comprising a PvuI restriction enzyme recognition site, resulting in nucleic acid molecules having one open and one closed end. Following the addition of a sequencing adapter to the open end, the nucleic acid molecules were sequenced on the nanopore deep-sequencing platform. A detailed overview of the experiment is provided below:

### λ DNA-HindIII Digest and Indexed Adapter Ligation

In this example, six different linkers were tested for construction of linked (or “closed”) adapters (see below under “Linked adapter ligation”). In order to multiplex sequence libraries prepared by each one of these six linked adapters, sample barcodes were added to the λ DNA-HindIII digest-fragments of each sample by using indexed adapters, wherein the index is used as sample barcode. For the preparation of each of the six different indexed adapters (Integrated DNA Technologies, Inc.), two single-stranded oligonucleotides were admixed at equimolar concentrations to form the top and bottom strand, respectively, of the following indexed double-stranded adapters:

Indexed adapter 1:
Top strand:
(SEQ ID NO: 11)
AAGGTTAACACAAAGACACCGACAACTTTCTTCAGCACCTC
Bottom strand:
(SEQ ID NO: 12)
AGCTGAGGTGCTGAAGAAAGTTGTCGGTGTCTTTGTGTTAACCTT
Indexed adapter 2:
Top strand:
(SEQ ID NO: 13)
AAGGTTAAACAGACGACTACAAACGGAATCGACAGCACCTC
Bottom strand:
(SEQ ID NO: 14)
AGCTGAGGTGCTGTCGATTCCGTTTGTAGTCGTCTGTTTAACCTT
Indexed adapter 3:
Top strand:
(SEQ ID NO: 15)
AAGGTTAACCTGGTAACTGGGACACAAGACTCCAGCACCTC
Bottom strand:
(SEQ ID NO: 16)
AGCTGAGGTGCTGGAGTCTTGTGTCCCAGTTACCAGGTTAACCTT
Indexed adapter 4:
Top strand:
(SEQ ID NO: 17)
AAGGTTAATAGGGAAACACGATAGAATCCGAACAGCACCTC
Bottom strand:
(SEQ ID NO: 18)
AGCTGAGGTGCTGTTCGGATTCTATCGTGTTTCCCTATTAACCTT
Indexed adapter 5:
Top strand:
(SEQ ID NO: 19)
AAGGTTAAAAGGTTACACAAACCCTGGACAAGCAGCACCTC
Bottom strand:
(SEQ ID NO: 20)
AGCTGAGGTGCTGCTTGTCCAGGGTTTGTGTAACCTTTTAACCTT
Indexed adapter 6
Top strand:
(SEQ ID NO: 21)
AAGGTTAAGACTACTTTCTGCCTTTGCGAGAACAGCACCTC
Bottom strand:
(SEQ ID NO: 22)
AGCTGAGGTGCTGTTCTCGCAAAGGCAGAAAGTAGTCTTAACCTT.

All sequences herein are indicated in the 5′-end to 3′-end direction.

After admixing at equimolar concentration, each of the indexed adapters were diluted in nuclease free water to a concentration of 4 pmol/μL. For indexed adapter ligation, 1 μg λ DNA-HindIII digest was used as starting material. Prior to indexed adapter ligation, to 2 μL of the λ DNA-HindIII digest, 38 μL 10 mMTris/HCl pH7.5 was added and incubated for 10 minutes at 85° C. and placed on ice. In Table 1 the reaction mixture per indexed adapter is indicated; for each type of indexed adapter, the reaction was performed in five-plex, rendering a total of 30 reaction mixtures.

TABLE 1
Reaction mixture for indexed adapter
ligation to λ DNA-HindIII digest.

| Reagent | Volume | |
|---|---|---|
| λ DNA-HindIII digest (25 ng/ul) | 40 | μL |
| (New England Biolabs; N3012; 500 ug/ml) | | |
| 10*CutSmart ® Buffer (New England Biolabs; B6004S) | 1 | μL |
| HindIII (New England Biolabs; R0104L, 20,000 units/ml) | 0.05 | μL |
| Adenosine 5′-Triphosphate (New England Biolabs; 100 mM, | 0.4 | μL |
| P0756) | | |
| Indexed adapter (4 pmol/μL) | 5 | μL |
| Ambion ™ - Nuclease Free Water (Invitrogen; AM9937) | 1.55 | μL |
| T4 DNA ligase (New England Biolabs; M0202, 400 U/μL) | 2 | μL |
| Total | 50 | μL |

The mixtures were incubated for 3 hours at 37° C. All five reaction mixtures per type of indexed adapter were pooled and purified by adding 2 volumes of AMPure PB beads according to manufacturer's instructions (Pacific Bio Science; 100-265-900) and eluted in 30 μL nuclease free water. The concentration of DNA was measured using the Qubit BR (Invitrogen, Qubit™ dsDNA BR Assay Kit, Q32853): Sample indexed with barcode 1:19.5 ng/μL (585 ng in 30 μL), Sample indexed with barcode 2:14.9 ng/μL (447 ng in 30 μL), Sample indexed with barcode 3:21.0 ng/μL (630 ng in 30 μL), Sample indexed with barcode 4:19.0 ng/μL (570 ng in 30 μL), Sample indexed with barcode 5:19.7 ng/μL (591 ng in 30 μL), Sample indexed with barcode 6:20.8 ng/μL (624 ng in 30 μL)

### Polishing/A-Addition

The indexed λ DNA HindIII digested fragments were polished and A-addition was performed by preparing a reagent mixture per indexed sample of Table 2:

TABLE 2
Reaction mixture for end polishing and A-addition
of indexed λ DNA-HindIII fragments.

| Reagent | Volume | |
|---|---|---|
| Indexed λ DNA HindIII fragments | 30 | μL |
| Ambion ™ - Nuclease Free Water (Invitrogen; AM9937) | 20 | μL |
| NEBNext FFPE DNA Repair Buffer | 3.5 | μL |
| (New England Biolabs; E6622AA) | | |
| Ultra II End-prep reaction buffer (New England Biolabs; | 3.5 | μL |
| E7647AA) | | |
| Flicking the tube and spin down before adding the following | | |
| reagents: | | |
| Ultra II End-prep enzyme mix (New England Biolabs; | 3 | μL |
| E7646AA) | | |
| NEBNext FFPE DNA Repair Mix (New England Biolabs; | 2 | μL |
| M6630) | | |
| Total | 62 | μL |

Using a thermal cycler, the reaction mixtures were incubate at 20° C. for 30 minutes followed by incubation at 65° C. for 30 minutes. Subsequently, the product was purified by adding 2 volumes of AMPure PB beads according to manufacturer's instructions (Pacific Bio Science; 100-265-900), and eluted in 10 μL nuclease free water.

### Linked Adapter Ligation

Six differently modified 5′-end phosphorylated single-stranded oligonucleotides were ordered at IDT to form (3′-T overhang) linked adapter no. 1-6, having the following structure (from 5′ to 3′):

### Linked Adapter 1:

GCAATACGTAACTGAACGAAGTACATT/iSpC3/AATGTACTTCGTTCAGTTACGTATTGCT, wherein GCAATACGTAACTGAACGAAGTACATT is indicated herein as SEQ ID NO: 23, wherein AATGTACTTCGTTCAGTTACGTATTGCT is indicated herein as SEQ ID NO: 24, and wherein the 3′-end T residue of SEQ ID NO: 23 is linked to the 5′-end A residue of SEQ ID NO: 24 by a C3 spacer (/iSpC3/);

### Linked Adapter 2:

GCAATACGTAACTGAACGAAGTACATT/iSpC9/AATGTACTTCGTTCAGTTACGTATTGCT, wherein GCAATACGTAACTGAACGAAGTACATT is indicated herein as SEQ ID NO: 25, wherein AATGTACTTCGTTCAGTTACGTATTGCT is indicated herein as SEQ ID NO: 26, and wherein the 3′-end T residue of SEQ ID NO: 25 is linked to the 5′-end A residue of SEQ ID NO: 26 by a C9 spacer (/iSpC9/);

### Linked Adapter 3:

GCAATACGTAACTGAACGAAGTACATT/iSpC18/AATGTACTTCGTTCAGTTACGTATTGCT, wherein GCAATACGTAACTGAACGAAGTACATT is indicated herein as SEQ ID NO: 27, wherein AATGTACTTCGTTCAGTTACGTATTGCT is indicated herein as SEQ ID NO: 28, and wherein the 3′-end T residue of SEQ ID NO: 27 is linked to the 5′-end A residue of SEQ ID NO: 28 by a C18 spacer (/iSpC18/);

### Linked Adapter 4:

GCAATACGTAACTGAACGAAGTACATT/idSp/AATGTACTTCGTTCAGTTACGTATTGCT, wherein GCAATACGTAACTGAACGAAGTACATT is indicated herein as SEQ ID NO: 29, wherein AATGTACTTCGTTCAGTTACGTATTGCT is indicated herein as SEQ ID NO: 30, and wherein the 3′-end T residue of SEQ ID NO: 29 is linked to the 5′-end A residue of SEQ ID NO: 30 by a 1′,2′-dideoxyribose spacer (/idSp/);

### Linked Adapter 5:

GCAATACGTAACTGAACGAAGTACATT/idSp/idSp/idSp/AATGTACTTCGTTCAGTTACGTATTG CT, wherein GCAATACGTAACTGAACGAAGTACATT is indicated herein as SEQ ID NO: 31, wherein AATGTACTTCGTTCAGTTACGTATTGCT is indicated herein as SEQ ID NO: 32, and wherein the 3′-end T residue of SEQ ID NO: 31 is linked to the 5′-end A residue of SEQ ID NO: 32 by three 1′,2′-dideoxyribose spacer moieties (/idSp/idSp/idSp/);

### Linked Adapter 6:

GCAATACGTAACTGAACGAAGTACATT (/idSp/idSp/idSp/idSp/idSp/) AATGTACTTCGTTCAGTT ACGTATTGCT, wherein GCAATACGTAACTGAACGAAGTACATT is indicated herein as SEQ ID NO: 33, wherein AATGTACTTCGTTCAGTTACGTATTGCT is indicated herein as SEQ ID NO: 34, and wherein the 3′-end T residue of SEQ ID NO: 33 is linked to the 5′-end A residue of SEQ ID NO: 34 by a five 1′,2′-dideoxyribose spacer moieties (/idSp/idSp/idSp/idSp/idSp/).

Each of these six modified oligonucleotides were diluted to a concentration of 50 μM as indicated in Table 3.

TABLE 3
Dilution of modified single-stranded oligonucleotides.

| Reagent | Volume | |
|---|---|---|
| Modified oligonucleotides (500 μM) | 2 | μL |
| Tris/HCl (50 mM) | 10 | μL |
| Ambion ™ - Nuclease Free Water (Invitrogen; AM9937) | 7.6 | μL |
| NaCl (5M) (Sigma-Aldrich; S7653) | 0.4 | μL |
| Total | 20 | μL |

The mixtures were heated to 90° C. for three minutes followed by slow cooling at 0.01° C. per second to 4° C. in a thermal cycler for controlled annealing, thereby allowing the modified oligonucleotides to fold back into a double-stranded structure comprising the linker on one end, i.e. to form a linked adapter. The linked adapters were diluted to 5 μM and ligated to the end-repaired and A-added-indexed fragments. Linked adapter 1 was ligated to the fragments comprising indexed barcode 1, linked adapter 2 was ligated to the fragments comprising indexed barcode 2, etcetera, by preparing a reaction mixture per indexed sample as indicated in Table 4.

TABLE 4
Reaction mixture for ligation of linked adapters to end
polished and A-added-indexed λ DNA HindIII fragments.

| Reagent | Volume | |
|---|---|---|
| End polished and A-added-indexed λ DNA-HindIII fragments | 10 | μL |
| 10X diluted linked adapter (5 μM) | 0.6 | μL |
| Blunt/TA Ligase Master Mix (New England Biolabs; M0367) | 10 | μL |
| Total | 20.6 | μL |

The reaction mixtures were incubated at 21° C. for 1 hour. The samples were purified by adding 2 volumes of AMPure PB beads according to manufacturer's instructions (Pacific Bio Science; 100-265-900), and eluted in 10 μL nuclease free water. The concentration of DNA was measured using the Qubit HS: Sample indexed barcode 1:27.6 ng/μL (276 ng in 10 μL), Sample indexed barcode 2:23.0 ng/μL (230 ng in 10 μL), Sample indexed barcode 3:32.0 ng/μL (320 ng in 10 μL), Sample indexed barcode 4:28.2 ng/μL (282 ng in 10 μL), Sample indexed barcode 5:35.2 ng/μL (352 ng in 10 μL), Sample indexed barcode 6:30.0 ng/μL (300 ng in 10 μL).

### ExoV Treatment

Each of the products of the linked adapter ligations was treated with Exonuclease V in order to remove non-closed polynucleotides. Per indexed sample, a reaction mixture was prepared as indicated in Table 5.

TABLE 5
Reaction mixture for exonuclease digestion
of non-closed polynucleotides.

| Reagent | Volume | |
|---|---|---|
| Linked adapter ligation product | 10.0 | μL |
| 10*NEBuffer ™ 4 (New England Biolabs; B7004) | 3.0 | μL |
| Adenosine 5′-Triphosphate (New England Biolabs; 100 mM, | 3.0 | μL |
| P0756) | | |
| ExoV (RecBCD) (New England Biolabs; 10,000 units/ml, | 2.0 | μL |
| M034) | | |
| Ambion ™ - Nuclease Free Water (Invitrogen; AM9937) | 12.0 | μL |
| Total | 30.0 | μL |

The reaction mixtures were incubate at 37° C. for 2 hours followed by incubation at 65° C. for 10 minutes. The samples were purified by adding 2 volumes of AMPure PB beads according to manufacturer's instructions (Pacific Bio Science; 100-265-900), and eluted in 20 μL nuclease free water. The DNA concentration of the samples was measured at the Qubit HS: Sample indexed barcode 1:3.66 ng/μL (73.2 ng in 20 μL; 26.5% of input), Sample indexed barcode 2:5.24 ng/μL (104.8 ng in 20 μL; 45.6% of input), Sample indexed barcode 3:5.04 ng/μL (100.8 ng in 20 μL; 31.5% of input), Sample indexed barcode 4:5.58 ng/μL (111.6 ng in 20 μL; 39.6% of input), Sample indexed barcode 5:5.58 ng/μL (111.6 ng in 20 μL; 31.7% of input), Sample indexed barcode 6:5.18 ng/μL (103.6 ng in 20 μL; 34.5% of input).

### PvuI Restriction

ExoV treated samples were treated with PvuI in order to open covalently closed HindIII fragments comprising a PvuI restriction site rendering polynucleotide fragments having an open and a closed end. Per indexed sample, a reaction mixture was prepared as indicated in Table 6:

TABLE 6
Reaction mixture PvuI digestion of closed fragments.

| Reagent | Volume | |
|---|---|---|
| Sample adapter product | 20.0 | μL |
| 10*NEBuffer ™ 3.1 (New England Biolabs; B7203) | 5.0 | μL |
| PvuI (New England Biolabs; R0150; 10,000 units/ml) | 1.0 | μL |
| Ambion ™ - Nuclease Free Water (Invitrogen; AM9937) | 24.0 | μL |
| Total | 50.0 | μL |

The samples were incubated for 1 hour at 37° C., purified by adding 2 volumes of AMPure PB beads according to manufacturer's instructions (Pacific Bio Science; 100-265-900), and eluted in 15 μL nuclease free water.

### Polishing/A-Addition

The PvuI restricted fragments were polished and A-addition was performed by preparing per indexed sample, a reagent mixture as indicated in Table 7.

TABLE 7
End polishing and A-addition of PvuI restricted fragments.

| Reagent | Volume | |
|---|---|---|
| PvuI restricted fragments | 15.0 | μL |
| Ambion ™ - Nuclease Free Water | 35.0 | μL |
| (Invitrogen; AM9937) | | |
| NEBNext FFPE DNA Repair Buffer | 3.5 | μL |
| (New England Biolabs; E6622AA) | | |
| Ultra II End-prep reaction buffer | 3.5 | μL |
| (New England Biolabs; E7647AA) | | |
| Flicking the tube and spindown before | | |
| adding the following reagents: | | |
| Ultra II End-prep enzyme mix | 3.0 | μL |
| (New England Biolabs; E7646AA) | | |
| NEBNext FFPE DNA Repair Mix | 2.0 | μL |
| (New England Biolabs; M6630) | | |
| Total | 62.0 | μL |

Using a thermal cycler, the reaction mixtures were incubate at 20° C. for 30 minutes followed by incubation at 65° C. for 30 minutes. Subsequently, the product was purified by adding 2 volumes of AMPure PB beads according to manufacturer's instructions (Pacific Bio Science; 100-265-900), and eluted in 7.5 μL nuclease free water.

### Native Barcode Ligation

After end repairing and A-addition, native barcoded adapters were ligated to the PvuI restricted fragments. Per sample, the native adapters comprised the identical barcodes as the indexing adapters, thereby rendering fragments comprising identical indices at the closed and the open ends of the fragments. For this, per indexed sample, a reagent mixtures was prepared as indicated in Table 8.

TABLE 8
native barcoded adapters were ligated
to the PvuI restricted fragments.

| Reagent | Volume | |
|---|---|---|
| End repaired and A-added opened fragments | 7.5 | μL |
| Native barcode(Oxford Nanopore | 2.5 | μL |
| Technologies; SQK-NBD112.24) | | |
| Blunt/TA Ligase Master Mix | 10.0 | μL |
| (New England Biolabs; M0367) | | |
| Total | 20 | μL |

The samples were incubated for 20 minutes at 21° C. Thereafter, 2 μL of EDTA (Oxford Nanopore Technologies; SQK-NBD112.24) was added and samples were mixed. Subsequently, the samples were pooled and the product was purified by adding 2 volumes of AMPure PB beads according to manufacturer's instructions (Pacific Bio Science; 100-265-900), and eluted in 12.5 μL nuclease free water.

### ONT Library Preparation

The pooled sample was prepared for ONT sequencing, using Ligation Sequencing Kit SQK-LSK112.24 (Oxford Nanopore Technologies). In a 1.5 ml Eppendorf DNA LoBind tube, a reaction mixture was prepared as indicated in Table 9.

TABLE 9
Reaction mixture for ONT sequencing.

| Reagent | Volume | |
|---|---|---|
| Pooled samples | 12.5 | μL |
| Ambion ™ - Nuclease Free Water (Invitrogen; AM9937) | 17.5 | μL |
| Quick Ligation reaction buffer | 10.0 | μL |
| (New England Biolabs; E6058AA) | | |
| Quick T4 DNA Ligase (New England Biolabs; E6057AA) | 5.0 | μL |
| Adapter Mix II H (AMXII-H) | 5.0 | μL |
| (ONT; Ligation Sequencing Kit SQK-LSK112.24) | | |
| Total | 50.0 | μL |

The sample was incubated at 20° C. for 10 minutes. Subsequently, the product was purified by adding 0.4 volumes AMpure PB beads according to manufacturer's ONT instructions (Oxford Nanopore Technologies; SQK-NBD112.24) using 250 μL Small Fragment Buffer (SFB) (Oxford Nanopore Technologies; SQK-NBD112.24) for washing the beads, and eluted in 15 μL EB (Oxford Nanopore Technologies; SQK-NBD112.24).

### Oxford Nanopore Technologies Sequencing and Data Processing

The processed sample was sequenced on a MinION Mk1B portable sequencing device (Oxford Nanopore Technologies) using flow cell R10.4 with real-time basecalling (MinKNOW v 22.05.5; (Oxford Nanopore Technologies) for 72 hours. After sequencing reads were re-basecalled using the SUP basecalling option in MinKNOW v 22.05.5 (Oxford Nanopore Technologies) to generate sequencing data with the highest accuracy.

Duplex sequence generation was performed using the command line version of Duplex Tools v0.2.20 (Oxford Nanopore Technologies) in which duplex reads were identified and prepared for basecalling and for recovering simplex basecalls from linked reads. Next, duplex reads were created using the command line version of the standard ONT basecall software.

### Results & Conclusion

Sequencing of the pooled barcoded samples resulted in a total of 976,000 reads corresponding to 0.6 gigabases of sequence. In total 424,727 reads (0.484 gigabases) of the sequencing data was assigned to one of the 6 used barcodes, i.e. de-multiplexed. For each of the resulting 6 barcoded data sets the amount of identified duplex sequences as percentage of the total amount of nucleotides using the command line version of the standard ONT basecall software (normal duplex analysis) and using the command line version of Duplex Tools v0.2.20 Split duplex sequencing (split duplex analysis), respectively. Results are shown in Table 10 below.

TABLE 10
Percentage of duplex sequenced nucleotides (in percentage
of total sequencing nucleotides generated) when
using normal or split duplex analysis.

| | Normal duplex | Split duplex |
|---|---|---|
| Modification | analysis: | analysis |
| iSpC3 | 4.91% | 25.11% |
| iSpC9 | 0.90% | 6.08% |
| iSpC18 | 2.66% | 15.30% |
| idSp | 6.22% | 27.99% |
| idSp-idSp-idSp | 6.68% | 28.82% |
| idSp-idSp-idSp-idSp-idSp | 0.88% | 3.26% |

A clear effect is observed related to the modification(s) present in the linked adapters. Highest percentages of duplex sequence nucleotides are observed when using a single or three consecutive “idSp” as linker. of the single idSp linker generated in total 34.21% of duplex sequence nucleotides, while the 3× idSp (idSp-idSp-idSp) linker generated 35.5% of duplex sequence nucleotides.

Results show that the linked adapter approach substantially increased the number of duplex reads up to ~35%, which results in a substantial increase in accuracy improvement.

### Example 2

In this example sgRNA-guided digestion with IDT Alt-R™ HiFi Cas9 Nuclease V3 (Integrated DNA Technologies, 1081058) was applied instead of restriction enzymes in the first fragmentation step, to generate more evenly distributed DNA fragments from 2 DNA. This allowed for improved comparison of duplex sequence generation between covalently closed and open linear DNA fragments of the same size. Lambda DNA was cut with Cas9RNP (Cas9-guideRNA ribo-protein complex) using the four sgRNAs depicted in Table 11.

TABLE 11
sgRNAs used for A DNA digestion.

| | | |
|---|---|---|
| sgRNA3 | TAATACGACTCACTATAGGctgcttcgtttatgcgctggGTTTTAGAGCT | SEQ ID NO: 35 |
| | AGAAATAGCAAGTTAAAATAAGGCTAGTCCGTTATCAACTTGA | |
| | AAAAGTGGCACCGAGTCGGTGCTTTTTTT | |
| sgRNA10 | TAATACGACTCACTATAGGcgccacgtctgcctccaccgGTTTTAGAGC | SEQ ID NO: 36 |
| | TAGAAATAGCAAGTTAAAATAAGGCTAGTCCGTTATCAACTTG | |
| | AAAAAGTGGCACCGAGTCGGTGCTTTTTTT | |
| sgRNA15 | TAATACGACTCACTATAGGgcgctttctactcgtgatttGTTTTAGAGCTA | SEQ ID NO: 37 |
| | GAAATAGCAAGTTAAAATAAGGCTAGTCCGTTATCAACTTGAA | |
| | AAAGTGGCACCGAGTCGGTGCTTTTTTT | |
| sgRNA21 | TAATACGACTCACTATAGGatttacaatatcgtcctgttGTTTTAGAGCTA | SEQ ID NO: 38 |
| | GAAATAGCAAGTTAAAATAAGGCTAGTCCGTTATCAACTTGAA | |
| | AAAGTGGCACCGAGTCGGTGCTTTTTTT | |

For sgRNA/Cas9 digestion, first gRNA-Cas9 ribonucleoprotein (RNP) complexes were formed, which were subsequently contacted with λ DNA. gRNA-Cas9 RNPs were formed by preparing a reaction mixture as indicated in Table 12 in fourteen-plex. For complex formation, the mixtures were incubated at room temperature for 10 minutes.

TABLE 12
Preparation of gRNA-Cas9 RNP complexes

| Reagent | Volume | |
|---|---|---|
| Ambion ™ - Nuclease Free Water (Invitrogen; AM9937) | | |
| 10*CutSmart ® Buffer (New England Biolabs; B6004S) | 5.92 | μL |
| sgRNAs (4plex 15.6 pmol/μL) | 3 | μL |
| IDT Alt-R ™ HiFi Cas9 Nuclease V3 (62 μM) | 0.86 | μL |
| (Integrated DNA Technologies; 62 μM, 62 1081058) | 0.22 | μL |
| Total: | 10 | μL |

For digestion with of λ DNA with gRNA-Cas9 RNPs, a reaction mixtures as described in Table 13 was prepared in fourteen-plex. The mixtures were incubated for 1 hour at 37° C. for digestion, followed by Cas9 inactivation for 10 minutes at 80° C.

TABLE 13
λ DNA digestion by Cas9RNP complex

| Reagent | Volume | |
|---|---|---|
| gRNA-Cas9 RNP | 10 | μL |
| λ DNA (50 ng/μL) (New England Biolabs; 500 ug/ml, N3011) | 20 | μL |
| Total | 30 | μL |

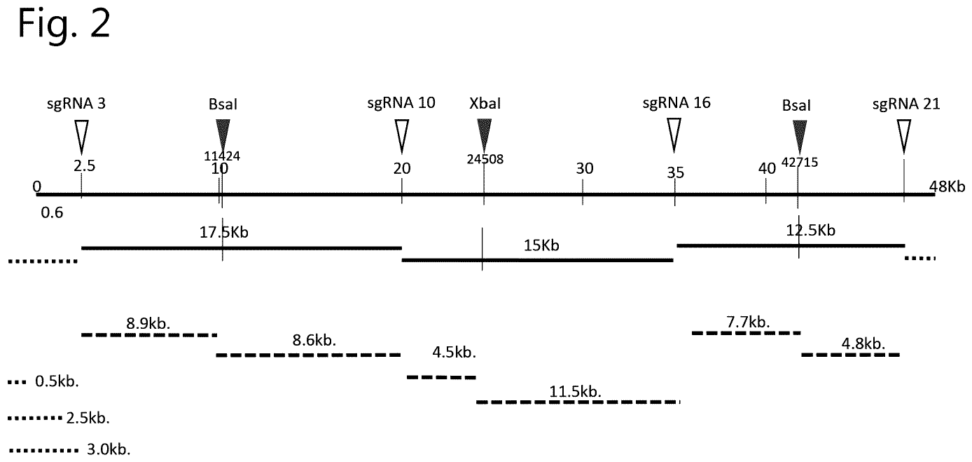

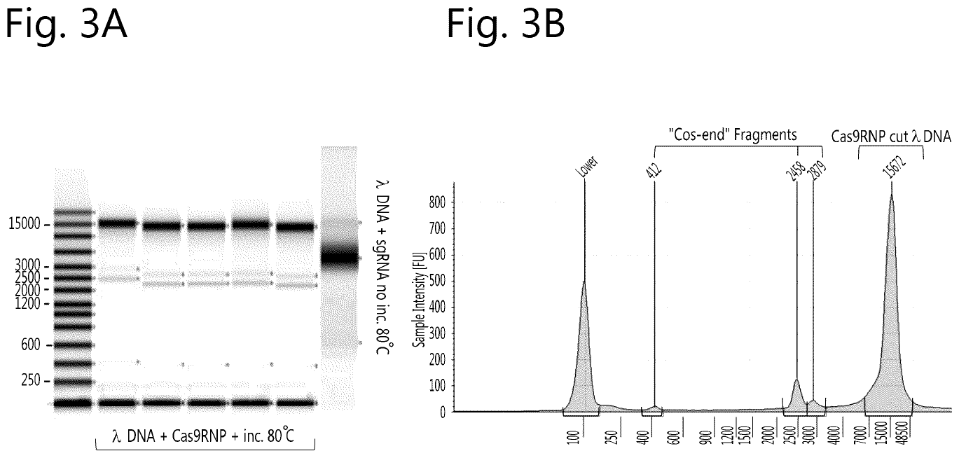

After in incubation, the fourteen-plex reaction mixtures were pooled, purified by adding 2 volumes of AMPure PB beads according to manufacturer's instructions (Pacific Bio Science; 100-265-900), and eluted in 94 μL nuclease free water. Digestion resulted in six λ DNA-fragments (three main fragments and three Cos-end fragments, of which the three main fragments each comprised a single restriction enzyme recognition site for BsaI or XbaI (New England Biolabs, R053 and R0145) as indicated in FIGS. 2 and 3. The purified Cas9RNP cut λ DNA was used as starting material for preparing the “first digest sample” starting with digestion by BsaI and XbaI (see Table 14), and as starting material for preparing the “second digest sample” starting with end-polishing and A addition (see Table 17), as described below.

### First Digest Sample

For BsaI and XbaI digestion of the Cas9RNP cut λ DNA, a reaction mixture as indicate in Table 14 was prepared in five-plex. The mixtures were incubated at 37° C. for 1 hour, followed by incubation at 65° C. for 10 minutes.

TABLE 14
First digest sample digestion by BsaI and XbaI

| Reagent | Volume | |
|---|---|---|
| Cas9RNP cut λ DNA (1 ug) | 8 | μL |
| BsaI (New England Biolabs; R0535, 10,000 units/ml) | 2 | μL |
| XbaI (New England Biolabs; R0145, 20,000 units/ml) | 1 | μL |
| 10*rCutSmart ™ Buffer (New England Biolabs; B6004) | 2.5 | μL |
| Ambion ™ -Nuclease Free Water (Invitrogen; AM9937) | 11.5 | μL |
| Total | 25 | μL |

After digestion, the five-plex reaction mixtures were pooled, purified by adding 2 volumes of AMPure PB beads according to manufacturer's instructions (Pacific Bio Science; 100-265-900), and eluted in 48 μL nuclease free water.

For end-polishing and A-addition of the digested fragments, a reaction mixture as indicated in Table 15 was prepared in five-plex.

TABLE 15
End polishing and A-addition of BsaI/XbaI cut λ DNA.

| Reagent | Volume | |
|---|---|---|
| BsaI/XbaI cut λ DNA | 8.0 | μL |
| Ambion ™ -Nuclease Free Water | 40.0 | μL |
| (Invitrogen; AM9937) | | |
| NEBNext FFPE DNA Repair Buffer | 3.5 | μL |
| (New England Biolabs; E6622AA) | | |
| Ultra II End-prep reaction buffer | 3.5 | μL |
| (New England Biolabs; E7647AA) | | |
| Flicking the tube and spindown before | | |
| adding the following reagents: | | |
| Ultra II End-prep enzyme mix | 3.0 | μL |
| (New England Biolabs; E7646AA) | | |
| NEBNext FFPE DNA Repair Mix | 2.0 | μL |
| (New England Biolabs; M6630) | | |
| Total | 60.0 | μL |

The reaction mixtures were incubate at 20° C. for 30 minutes followed by incubation at 65° C. for 30 minutes. After end-polishing and A-addition, the five-plex reaction mixtures were pooled, purified using 1 volume AMpure clean up, and eluted in 61 μL nuclease free water.

Sequencing adapters were ligated to the end polished and A-added BsaI/XbaI cut 2 DNA fragments in order to prepare the sample for ONT sequencing, using Ligation Sequencing Kit SQK-LSK114 (Oxford Nanopore Technologies). For this, a reaction mixture was prepared as indicated in Table 16 in a 1.5 ml Eppendorf DNA LoBind tube.

TABLE 16
Reaction mixture for ONT SQK-LSK114
sequencing adapter ligation.

| Reagent | Volume | |
|---|---|---|
| Pooled sample | 60 | μL |
| Ligase buffer (LNB) (ONT; Ligation | 25 | μL |
| Sequencing Kit SQK-LSK114) | | |
| Quick T4 DNA Ligase | 10 | μL |
| (New England Biolabs; E6057AA) | | |
| Ligation adapter (LA) (ONT; Ligation | 5 | μL |
| Sequencing Kit SQK-LSK114) | | |
| Total | 100 | μL |

The mixture was incubated at 20° C. for 20 minutes. Subsequently, the product was purified by adding 0.4 volumes AMpure PB beads according to manufacturer's ONT instructions (Oxford Nanopore Technologies; SQK-LSK114) using 250 μL Small Fragment Buffer (SFB) (Oxford Nanopore Technologies; SQK-LSK114) for washing the beads, and eluted in 15 μL EB (Oxford Nanopore Technologies; SQK-LSK114).

### The Second Digest Sample

For end-polishing and A-addition of gRNA-Cas9 RNP cut 2 DNA, a reaction mixture as indicated in Table 17 was prepared in five-plex.

TABLE 17
End polishing and A-addition of the second digest sample.

| Reagent | Volume | |
|---|---|---|
| gRNA-Cas9 RNP cut λ DNA (1 ug) | 8.0 | μL |
| Ambion ™ -Nuclease Free Water | 40.0 | μL |
| (Invitrogen; AM9937) | | |
| NEBNext FFPE DNA Repair Buffer | 3.5 | μL |
| (New England Biolabs; E6622AA) | | |
| Ultra II End-prep reaction buffer | 3.5 | μL |
| (New England Biolabs; E7647AA) | | |
| Flicking the tube and spindown before | | |
| adding the following reagents: | | |
| Ultra II End-prep enzyme mix | 3.0 | μL |
| (New England Biolabs; E7646AA) | | |
| NEBNext FFPE DNA Repair Mix | 2.0 | μL |
| (New England Biolabs; M6630) | | |
| Total | 60.0 | μL |

The reaction mixtures were incubate at 20° C. for 30 minutes followed by incubation at 65° C. for 30 minutes. Subsequently, the product was pooled, purified by adding 2 volumes of AMPure PB beads according to manufacturer's instructions (Pacific Bio Science; 100-265-900), and eluted in 40 μL nuclease free water.

Subsequently, these fragments were covalently closed using idSp-linked adapters. Before ligation, the linked adapter was diluted in a mixture as indicated in Table 18 and denatured for 3 minutes at 90°, followed by immediate intra-molecular renaturation of the palindromic sequence on ice (0° C.).

TABLE 18
Denaturation/renaturation of idSp linked-adapter mixture

| Reagent | Volume | |
|---|---|---|
| Linked adapter (500 μM) | 2 | μL |
| Tris/HCl (50 mM) | 10 | μL |
| NaCl (5M) (Sigma-Aldrich; S7653) | 0.4 | μL |
| Ambion ™ -Nuclease Free Water (Invitrogen; AM9937) | 7.6 | μL |
| Total | 20 | μL |

Then, ligation was performed with Blunt/TA Ligase Master Mix (New England Biolabs, M0367) and the Linked adapter 4 of Example 1 was ligated to covalently close the fragments at their unprotected ends. For this, a reaction mixture as indicated in Table 19 was prepared in five-plex.

TABLE 19
Ligation of linker-adaptor to gRNA-Cas9 RNP-cut λ DNA

| Reagent | Volume | |
|---|---|---|
| gRNA-Cas9 RNP-cut λ DNA (900 ng) | 8 | μL |
| 10X diluted idSp linked adapter mixture (5 μM) | 1.2 | μL |
| Blunt/TA Ligase Master Mix (New England Biolabs; M0367) | 10 | μL |
| Ambion ™ - Nuclease Free Water (Invitrogen; AM9937) | 0.8 | μL |
| Total | 20 | μL |

The mixtures was incubated at 21° C. for 1 hour, followed by pooling, purification using 2 volumes AMpure clean up, and elution in 30 L nuclease free water. Subsequently, a reaction mixture as indicated in Table 20 was prepared in order to remove any DNA fragments with open ends by exonuclease digestion.

TABLE 20
Reaction mixture for Exonuclease V digestion
of non-closed polynucleotides.

| Reagent | Volume | |
|---|---|---|
| Linked adapter ligation product (1 μg) | 10.0 | μL |
| 10*rCutSmart Buffer ® | 4.0 | μL |
| (New England Biolabs; B6004) | | |
| Adenosine 5′-Triphosphate | 4.0 | μL |
| (New England Biolabs; 100 mM, P0756) | | |
| DTT (10 mM)(Qiagen; 1M, 1032395) | 4.0 | μL |
| Exonuclease V (RecBCD) | 3.0 | μL |
| (New England Biolabs; M034, 10,000 units/ml) | | |
| Ambion ™ - Nuclease Free Water | 15.0 | μL |
| (Invitrogen; AM9937) | | |
| Total | 40.0 | μL |

The mixture was incubated at 37° C. for 90 minutes followed by 65° C. for 10 minutes. The product is AMpure (2 volumes) purified and eluted in 10 μL nuclease free water. Next, the three covalently closed fragments were opened again by digestion with a mixture of BsaI an XbaI (Table 21).

TABLE 21
Reaction mixture to cut covalently closed λ DNA open.

| Reagent | Volume | |
|---|---|---|
| Covalently closed λ DNA (314 ng) | 10.0 | μL |
| 10*rCutSmart Buffer ® (New England Biolabs; B6004) | 2.5 | μL |
| Xba I (New England Biolabs; R0145, 20,000 units/ml) | 1.0 | μL |
| Bsa I (New England Biolabs; R0535, 10,000 units/ml) | 2.0 | μL |
| Ambion ™ - Nuclease Free Water (Invitrogen; AM9937) | 9.5 | μL |
| Total | 25.0 | μL |

The mixture was incubated at 21° C. for 1 hour; then inactivate 65° C. for 10 min. The samples are AMpure (2 volumes) purified and eluted in 10 μL nuclease free water. Polishing and A-addition of the open fragments was done according to Table 17. After another round of 1 volume AMpure clean up, an elution in 61 μL nuclease free water, the fragments were prepared for ONT sequencing, using Ligation Sequencing Kit SQK-LSK114 (Oxford Nanopore Technologies). In a 1.5 ml Eppendorf DNA LoBind tube, a reaction mixture was prepared as indicated in Table 16 for sequencing-adapter ligation. The sample was incubated at 20° C. for 20 minutes. Subsequently, the product was purified by adding 0.4 volumes AMpure PB beads according to manufacturer's ONT instructions (Oxford Nanopore Technologies; SQK-LSK114) using 250 μL Small Fragment Buffer (SFB) (Oxford Nanopore Technologies; SQK-LSK114) for washing the beads, and eluted in 15 μL EB (Oxford Nanopore Technologies; SQK-LSK114).

Library preps from the two λ DNA samples (the first and the second digest sample) both comprise similar sized fragments amenable for sequencing, where the first sample is expected to generate predominantly simplex sequence reads (non-linked fragments), while the second sample is expected to generate an increased amount of duplex reads due to covalently closure of the fragments from one side (linked fragments).

### Oxford Nanopore Technologies Sequencing and Data Processing

Oxford Nanopore Technologies sequencing and data processing was performed as indicated in Example 1, i.e. both normal duplex analysis and split duplex analysis were performed on the non-linked as well as the linked fragment sequence libraries.

TABLE 22
Total number of reads, nucleotides and percentage nucleotides of total
duplex sequenced when using normal, split duplex analysis or both.

| | | Non-linked fragments | Linked fragments |
|---|---|---|---|
| | # nt in input file | 38,050,000,000 | 18,340,000,000 |
| Normal duplex | # duplex reads | 759491 | 220321 |
| analysis | # duplex nts | 4,271,513,379 | 1,462,697,389 |
| | % duplex nts | 11.23% | 7.98% |
| Split duplex | # duplex reads | 99802 | 1231476 |
| analysis | # duplex nts | 468,816,645 | 6,918,520,532 |
| | % duplex nts | 1.23% | 37.72% |
| Total | # duplex nts | 4,740,330,024 | 8,381,217,921 |
| | % duplex nts | 12.46% | 45.70% |
| | # simplex nts | 28,569,339,952 | 1,577,564,158 |

### Results and Conclusion

The results are summarized in Table 22. The non-linked fragment library yielded around 38 Gb of sequence nucleotide, while the linked fragment library produced 18, 34 sequence nucleotides. The amount of normal duplex reads decreased from 11.23% in the non-linked library to 7.98% in the linked fragment library. However, we observe a very strong increase of split duplex reads from 1.23% in the non-linked fragment library to 37.72% in the linked fragment library. In total, we observe 47% of duplex sequence reads in the linked fragment library, which approaches the absolute maximum of duplex sequence reads (50%) that can be theoretically achieved. The fact that most of these duplex reads are identified by the split duplex analysis tool provides compelling evidence that these duplex reads are generated as a result of physically linked fragments. This proofs that covalently closing DNA fragments for duplex sequencing is highly efficient using the linked adapter described in the invention.

### Example 3

A sequencing library using the adapters of the invention was used to show that the method as detailed herein indeed increases the accuracy of sequencing at the MGI DNBSEQ-G400 technology platform. PCR marker fragments (New England Biolabs; N3234; 300 μg/ml) were prepared as template DNA.

In short, linked adapters having a covalently closed end and linked adapters having a covalently closed end with a restriction enzyme site (NotI: GCGGCCGC) were added in the ratio 5:1 for ligation to both ends of PCR marker fragments, resulting in covalently closed nucleic acid fragments. By ExoV treatment any non-closed nucleic acids were removed. Subsequent NotI-HF® digestion resulted in opening of one end of the covalently closed PCR marker fragments, resulting in nucleic acid molecules having one open and one closed end. Following the addition of a phosphorylated sequencing adapter to the open end, the nucleic acid molecules were prepared for sequencing on the MGI DNBSEQ-G400 technology platform. A detailed overview of the experiment is provided below:

Prior to Polishing/A-addition, 220 μL of the PCR marker fragments (50 bp, 150 bp, 300 bp, 500 bp and 766 bp fragments) were purified by adding 1.5 volume of AMPure PB beads according to manufacturer's instructions (Pacific Bio Science; 100-265-900) and eluted in 111 μL nuclease free water. The concentration of DNA was measured using the Qubit BR (Invitrogen; Qubit™ 1x dsDNA BR Assay Kit Cat. Nr.: Q33266): PCR Marker fragments: 470 ng/μL (51.7 μg in 110 μL).

### Polishing/A-Addition

The PCR marker fragments were polished and A-addition was performed by preparing the reagent mixture of Table 23 per sample (30 samples).

TABLE 23
Reaction mixture for end polishing and
A-addition of PCR marker fragments.

| Reagent | Volume | |
|---|---|---|
| PCR marker (New England Biolabs; N3234) | 2.2 | μL |
| Ambion ™ - Nuclease Free Water (Invitrogen; AM9937) | 45.8 | μL |
| NEBNext FFPE DNA Repair Buffer (New England Biolabs; | 3.5 | μL |
| E6622AA) | | |
| Ultra II End-prep reaction buffer (New England Biolabs; | 3.5 | μL |
| E7647AA) | | |
| Flicking the tube and spin down before adding the following | | |
| reagents: | | |
| Ultra II End-prep enzyme mix (New England Biolabs; | 3 | μL |
| E7646AA) | | |
| NEBNext FFPE DNA Repair Mix (New England Biolabs; | 2 | μL |
| M6630) | | |
| Total | 60 | μL |

Using a thermal cycler, the reaction mixtures were incubated at 20° C. for 30 minutes followed by incubation at 65° C. for 30 minutes. Subsequently, the samples were pooled and precipitated by adding 2 volumes of 100% Ethanol (J. T Baker; Cat. Nr.: 8025.1000) and 1M NaAc (Sigma Aldrich Cat. No.: 25022-2.5 KG-R) to a final concentration of 0.1M NaAc. The product was washed using 1 volume of 70% EtOH and eluted in 46 μL nuclease free water. The concentration of the PCR Marker fragments was measured using the Qubit™ 1× dsDNA BR Assay Kit (Invitrogen; Q33266): End polished and A-added PCR Marker fragments: 567 ng/μL (25.5 μg in 45 μL)

### Adapters.

The first linked adapter used in this experiment is “linked adapter 4” as indicated herein above for example 1 (wherein the spacer is 1′,2′-dideoxyribose spacer (/idSp/)).
The second linked adapter used in this experiment comprised a NotI restriction site (underlined):

GCAATACGTAACTGAACGAAGTACATT/idSp/AATGT
ACTTCGTTCAGTTACGTATTGCGCGGCCGCT,

wherein GCAATACGTAACTGAACGAAGTACATT is indicated herein as SEQ ID NO: 39,
wherein AATGTACTTCGTTCAGTTACGTATTGCGCGGCCGCT is indicated herein as SEQ ID NO: 40,
and wherein the 3′-end T residue of SEQ ID NO: 39 is linked to the 5′-end A residue of SEQ ID NO: 40 by a 1′,2′-dideoxyribose spacer (/idSp/).

Each of these modified single-stranded oligonucleotides were diluted to a concentration of 50 μM as indicated in Table 24.

### Preparation Adapters

TABLE 24
Dilution of modified single-stranded oligonucleotides.

| Reagent | Volume | |
|---|---|---|
| Modified oligonucleotides (500 μM) | 2 | μL |
| Tris/HCl (50 mM) | 10 | μL |
| Ambion ™ - Nuclease Free Water (Invitrogen; AM9937) | 7.6 | μL |
| NaCl (5M) (Sigma-Aldrich; S7653) | 0.4 | μL |
| Total | 20 | μL |

The mixture was heated to 90° C. for three minutes followed by slow cooling at 0.01° C. per second to 4° C. in a thermal cycler for controlled annealing, thereby allowing the modified oligonucleotides to fold back into a double-stranded structure comprising the linker on one end, i.e. to form a linked adapter.

### Ligation Linked Adapters

Linked adapters (50 μM) were ligated to the end-repaired and A-added fragmented PCR Marker fragments, using a ten-fold excess. Reaction mixture was prepared as indicated in Table 25.

TABLE 25
Reaction mixture for ligation of linked adapters
to End polished and A-added PCR Marker fragments.

| Reagent | Volume | |
|---|---|---|
| End polished and A-added PCR Marker fragments | 1.0 | μL |
| First linked adapter (without NotI restriction site) (50 μM) | 2.7 | μL |
| Second linked adapter (including NotI restriction site) (50 μM) | 0.54 | μL |
| Ambion ™ - Nuclease Free Water (Invitrogen; AM9937) | 5.76 | μL |
| Blunt/TA Ligase Master Mix (New England Biolabs; M0367) | 10 | μL |
| Total | 20.0 | μL |

The reaction mixture was incubated at 21° C. for 1 hour. The samples were pooled, purified by adding 2 volumes of AMPure PB beads according to manufacturer's instructions (Pacific Bio Science; 100-265-900), and eluted in 31 L nuclease free water. The concentration of the Linked adapter ligation product was measured using the Qubit 1x dsDNA BR Assay Kit (Invitrogen, Q33266): Linked adapter ligation product: 206 ng/μL (6.2 μg in 30 μL)

### ExoV Treatment

In order to remove non-closed polynucleotides, the product of linked adapter ligation was treated with Exonuclease V in eight-fold. Reaction mixture was prepared as indicated in Table 26.

TABLE 26
Reaction mixture for Exonuclease V digestion
of non-closed polynucleotides.

| Reagent | Volume | |
|---|---|---|
| Linked adapter ligation product | 3.5 | μL |
| 10*rCutSmart Buffer ® (New England Biolabs; B6004) | 4.0 | μL |
| Adenosine 5′-Triphosphate (10 mM)(New England Biolabs; | 4.0 | μL |
| P0756) | | |
| DTT (10 mM)(Qiagen; 1M, 1032395) | 4.0 | μL |
| Exonuclease V (RecBCD) (New England Biolabs; M034, | 3.0 | μL |
| 10,000 units/ml) | | |
| Ambion ™ - Nuclease Free Water (Invitrogen; AM9937) | 21.5 | μL |
| Total | 40.0 | μL |

The reaction mixtures were incubate at 37° C. for 1.5 hours followed by incubation at 65° C. for 10 minutes. The samples were pooled, purified by adding 2 volumes of AMPure PB beads according to manufacturer's instructions (Pacific Bio Science; 100-265-900), and eluted in 26 μL nuclease free water. The concentration of the ExoV treated PCR Marker product was measured using the Qubit™ 1x dsDNA HS Assay Kit (Invitrogen: Cat. No.: Q33231): ExoV treated PCR Marker product: 145 ng/μL (3.63 μg in 25 μL)

### NotI-HF® Restriction

ExoV treated PCR Marker product was treated with NotI-HF® in five-fold in order to open covalently closed PCR Marker fragments comprising a NotI-HF® restriction site, rendering polynucleotide fragments having an open and a closed end. Reaction mixture was prepared as indicated in Table 27.

TABLE 27
Reaction mixture NotI-HF ® digestion of closed fragments.

| Reagent | Volume | |
|---|---|---|
| Sample ExoV treated product | 4.0 | μL |
| 10*rCutSmart Buffer ® (New England Biolabs; B6004) | 2.5 | μL |
| NotI-HF ® (New England Biolabs; R3189; 20,000 units/ml) | 2.0 | μL |
| Ambion ™ - Nuclease Free Water (Invitrogen; AM9937) | 16.5 | μL |
| Total | 25.0 | μL |

The samples were incubated for 1 hour at 37° C. followed by incubation at 65° C. for 20 minutes, pooled, purified by adding 2 volumes of AMPure PB beads according to manufacturer's instructions (Pacific Bio Science; 100-265-900), and eluted in 21 μL nuclease free water. The concentration of the NotI-HF® restricted PCR Marker product was measured using the Qubit™ 1x dsDNA HS Assay Kit (Invitrogen; Cat. No: Q33231): NotI-HF® restricted PCR Marker product: 123 ng/μL (2.46 μg in 20 μL).

### Preparing NotI-HF® Restricted PCR Marker Product for Making MGI DNA Nanoballs (DNBs)

The NotI-HF® restricted PCR Marker product was prepared for MGI DNBs making, using kit modules of the MGIEasy FS DNA Library Prep Set, Version 2.1 (MGI; Cat. No.: 1000006987), according to manufacturer's instructions (MGIEasy FS DNA Library Prep Set User Manual Version B4).

Preparation Custom (y-Shaped) MGI Adapter

5′-phosphorylated Index Custom oligonucleotide (index underlined) (top) in the 5′ to 3′ direction:

(SEQ ID NO: 41)
AGTCGGAGGCCAAGCGGTCTTAGGAAGACAATAGGTCC
GATCAACTCCTTGGCTCACA

Custom (5′-phosphorylated) oligonucleotide (bottom) in the 5′ to 3′ direction:

(SEQ ID NO: 42)
GAACGACATGGCTACGATCCGACTT

The custom 5′-phosphporylated single-stranded MGI oligonucleotides were annealed as indicated in Table 28.

TABLE 28
Annealing of custom single-stranded oligonucleotides.

| Reagent | Volume | |
|---|---|---|
| Index Custom oligonucleotide_top (100 μM) | 35 | μL |
| Custom oligonucleotide_bottom (100 μM) | 35 | μL |
| Tris/HCl (1M) | 1.75 | μL |
| NaCl (5M) (Sigma-Aldrich; S7653) | 1.4 | μL |
| Total | 73.15 | μL |

These mixture was heated to 90° C. for three minutes followed by fast cooling on ice.

### End Repair and A-Tailing

The NotI-HF® restricted PCR Marker fragments were End-repaired and A-addition was performed by preparing the reagent mixture of Table 29, according to manufacturer's instructions (Chapter 3 Library Construction Protocol; Section 3.3 End Repair and A-tailing, of the MGIEasy FS DNA Library Prep Set User Manual version B4).

TABLE 29
Reaction mixture for End Repair and A-tailing of NotI-
HF ® restricted PCR Marker fragments.

| Reagent | Volume | |
|---|---|---|
| NotI-HF ® restricted PCR Marker fragments | 4.0 | μL |
| TE Buffer | 36 | μL |
| ERAT Buffer (MGIEasy FS DNA Library Prep Kit; | 7.1 | μL |
| MGI; Cat. No.: 1000005254) | | |
| ERAT Enzyme Mix (MGIEasy FS DNA Library Prep Kit; | 2.9 | μL |
| MGI; Cat. No.: 1000005254) | | |
| Total | 50.0 | μL |

Using a thermal cycler, the sample was incubated for 30 minutes at 37° C. followed by incubation at 65° C. for 15 minutes.

### Custom MGI Adapter Ligation

To end repaired and A-tailed PCR Marker fragments the custom adapter was ligated by preparing the reagent mixture of Table 30, according to manufacturer's instructions (Chapter 3 Library Construction Protocol; Section 3.4 Adapter Ligation, of the MGIEasy FS DNA Library Prep Set User Manual Version B4).

TABLE 30
Reaction mixture for modified MGI Adapter ligation.

| Reagent | Volume | |
|---|---|---|
| End repaired and A-tailed PCR Marker fragments | 50 | μL |
| Ligation Buffer (MGIEasy FS DNA Library Prep Kit; | 23.4 | μL |
| MGI; Cat. No.: 1000005254) | | |
| DNA Ligase (MGIEasy FS DNA Library Prep Kit; | 1.6 | μL |
| MGI; Cat. No.: 1000005254) | | |
| Custom MGI Adapter (50 μM) | 2.7 | μL |
| Ambion ™ - Nuclease Free Water (Invitrogen; AM9937) | 2.3 | μL |
| Total | 80.0 | μL |

The sample was incubated for 30 minutes at 37° C. The product was cleaned up by adding 20 μL TE Buffer and 200 μL AMPure XP Beads (AMPure XP Bead-Based Reagent; Beckman Coulter; Cat. No.: A63881) according to manufacturer's instructions (Chapter 3 Library Construction Protocol; Section 3.5 Adapter Ligation, of the MGIEasy FS DNA Library Prep Set User Manual version B4), and eluted in 24 UL TE Buffer (Ambion, Cat. No. AM9858). The concentration of the product was measured using the Qubit™ 1× dsDNA HS Assay Kit (Invitrogen; Cat. No.: Q33231): Custom MGI adapter ligation product: 14.3 ng/μL (329 ng in 23 μL).

### Denaturation and Single Strand Circularization

To 23 μL Custom MGI adapter ligation product, 25 UL TE buffer (Ambion, Cat. No. AM9858) was added. In a thermocycler the product was heated at 95° C. for 3 minutes. After the reaction was completed the product was placed on ice for 2 minutes and centrifuged briefly. To denaturized PCR Marker ligation product, Single Strand Circulation was performed according to manufacturer's instructions (Chapter 3 Library Construction Protocol; Section 3.10 Single Strand Circularization, of the MGIEasy FS DNA Library Prep Set User Manual Version B4).

TABLE 31
Reaction mixture for Single Strand Circularization.

| Reagent | Volume | |
|---|---|---|
| Denaturized PCR Marker ligation product | 48 | μL |
| Splint Buffer (MGIEasy Circularization Module; | 11.6 | μL |
| MGI; Cat. No.: 1000005260) | | |
| DNA Rapid Ligase (MGIEasy Circularization Module; | 0.5 | μL |
| MGI; Cat. No.: 1000005260) | | |
| Total | 50.1 | μL |

The sample was incubated for 30 minutes at 37° C. in a thermocycler and placed on ice.

### Enzymatic Digestion

The circularized product was enzymatic digested according to manufacturer's instructions (Chapter 3 Library Construction Protocol; Section 3.11 Enzymatic Digestion, of the MGIEasy FS DNA Library Prep Set User Manual Version B4).

TABLE 32
Reaction mixture for Enzymatic Digestion.

| | |
|---|---|
| Reagent | Volume |

| | | |
|---|---|---|
| Circularized PCR Marker product | 50 | μL |
| Digestion Buffer (MGIEasy Circularization Module; | 1.4 | μL |
| MGI; Cat. No.: 1000005260) | | |
| Digestion Enzyme (MGIEasy Circularization Module; | 2.6 | μL |
| MGI; Cat. No.: 1000005260) | | |
| Total | 54 | μL |

In a thermocycler the sample was incubated at 37° C. for 30 minutes, subsequently 7.5 μL Digestion Stop Buffer (MGIEasy Circularization Module; MGI; Cat. No.: 1000005260) was added. The solution is transferred to a fresh 1.5 mL centrifuge tube and 170 L of AMPure XP Beads (AMPure XP Bead-Based Reagent; Beckman Coulter; Cat. No.: A63881) is added to the Enzymatic Digestion product. Cleanup is done according to manufacturer's instructions (Chapter 3 Library Construction Protocol; Section 3.12 Enzymatic Digestion Product Cleanup, of the MGIEasy FS DNA Library Prep Set User Manual Version B4). The concentration of the purified Enzymatic Digestion product was analysed with Qubit™ ssDNA Assay Kit (Invitrogen; Cat. No.: Q10212). Enzymatic Digestion product (ssDNA library): 13.6 ng/μL (408 ng in 30 μL).

### Making DNA Nanoballs (DNBs)

Enzymatic digestion product was prepared for sequencing at the MGI DNBSEQ—G400 technology platform, according to manufacturer's instructions (High-throughput (Rapid) Sequencing set—DNBSEQ-G400RS User Manual version 8.0) (FCS PE150, MGI; Cat. No.: 1000016982). Reaction mixture (1) was made as shown in Table 33.

TABLE 33
Reaction mixture (1) for making DNBs.

| Reagent | Volume | |
|---|---|---|
| ssDNA library | 5.3 | μL |
| Low TE Buffer | 14.7 | μL |
| Make DNB Buffer | 20 | μL |
| Total | 40 | μL |

The mix is placed into a thermalcycler and the hybridization reaction was performed using the thermal cycler settings shown in Table 34.

TABLE 34
Primer hybridization reaction condition.

| Temperature | Time |
|---|---|
| Heated lid (105° C.) | On |
| 95° C. | 1 min |
| 65° C. | 1 min |
| 40° C. | 1 min |
| 4° C. | Hold |

Subsequently DNB mixture 2 was made as shown in Table 35:

TABLE 35
Reaction mixture (2) for making DNBs.

| Reagent | Volume |
|---|---|
| Reaction mixture ssDNA library | 40 μL |
| Make DNB High-efficiency Enzyme Mix I | 40 μL |
| Make DNB Enzyme Mix II (LC) | 4 μL |
| Total | 88 μL |

The mix is placed into a thermal cycler and the hybridization reaction was performed using the thermal cycler settings shown in Table 36:

TABLE 36
Rolling circle amplification conditions.

| Temperature | Time |
|---|---|
| Heated lid (35° C.) | On |
| 30° C. | 25 min |
| 4° C. | Hold |

The reaction is stopped by adding 20 μL Stop DNB Reaction Buffer. The concentration of the product was measured using Qubit™ ssDNA Assay Kit (Invitrogen; Cat. No.: Q10212): DNBs Library: 3.33 ng/μL

### DNB Loading

DNB loading was performed according to manufacturer's instructions (chapter 6.2.2 MGIDLR-200RS DNB loading, of the High-throughput (Rapid) Sequencing set-DNBSEQ-G400RS User Manual version 8.0). To prepare the flow cell for DNB loading, the flow cell was balanced at room temperature for 60 minutes to 24 hours. DNB loading mix was made as shown in table 37.

TABLE 37
DNB loading mix.

| Reagent | Volume | |
|---|---|---|
| DNB load buffer II | 8 | μL |
| Make DNB Enzyme Mix II (LC) | 0.25 | μL |
| DNB | 25 | μL |
| Total | 33.25 | μL |

A sealing gasket and balanced flow cell were placed in the MGIDL-200H Portable DNB Loader and 30 μL DNB was added into the fluidics inlet. The flow cell was left on the bench for 30 minutes before use.

### Preparing the Sequencing Reagent Cartridge, Sequencing and Data-Processing

The processed sample was sequenced on DNBSEQ-G400RS (Cat. Nr.900-0000493-00) according to manufacturer's instructions (High-throughput (Rapid) Sequencing set-DNBSEQ-G400RS User Manual version 8.0). The sequencing reagent cartridge was prepared according to manufacturer's instructions (chapter 7 Preparing the sequencing reagent cartridge, of the High-throughput (Rapid) Sequencing set-DNBSEQ-G400RS User Manual version 8.0). The parameters for sequencing on de MGI-G400 were set according to manufacturer's instructions (chapter 8 Preparing the sequencing reagent cartridge, of the High-throughput (Rapid) Sequencing set-DNBSEQ-G400RS User Manual version 8.0). Demultiplexing was performed offline according to manufacturer's instructions (SplitBarcode Manual, version 2.0).

### Results & Conclusion

Sequencing reads obtained by paired-end sequencing were paired, i.e. read 1 (obtained by sequencing the DNB and comprising the following elements in the 5′- to 3′-direction: the PCR marker fragment, the first half of the linked-adapter sequence, the linker, the second half of the linked-adapter sequence in reversed orientation and the PCR marker fragment in reversed orientation) and read 2 (obtained by sequencing the strand copied from the DNB by a strand displacement polymerase and comprising the same sequence as read 1 were compared and a consensus read was constructed by selecting the called bases with the highest quality (highest Qvalue or Phred quality score) from read 1 and read 2. It was observed that at the position of the modification used in the linked adapter, an “A” or “T” nucleotide was incorporated by the a strand displacement polymerase used for the DNA-Ball generation.

Validation of each of the reads and the consensus reads with the known adapter sequence (see Table 38) learned that the resulting consensus sequence was of higher quality than the two separate single reads. In fact, all miscalls could be overcome by selecting from each of the reads the basecall with the highest Qvalue or Phred quality score, as shown in Table 38, columns “C”.

As indicated above, each of the separate reads (read 1 and 2) also comprises a duplex of the PCR fragment sequence and the linker-adapter sequence, albeit in reverse complement orientation after the linker. Hence, these duplex base calls within each of these separate reads can also be used to increase the quality of these single reads. In fact, all miscalls within each read could be overcome by selecting the called bases with the highest Qvalue or Phred quality score (Table 39) from these duplex sequences within a read, proofing that the present invention allows for high quality single reads and making paired end sequencing obsolete, taken that read length is sufficient for sequencing the full duplex sequences.

Hence in conclusion, a linker can be used for generating high quality duplex reads (within a read in case of single-end reading or also between reads in case of paired-end reading) using the MGI sequencing platform. The method described herein can thus increase sequencing (or “base calling”) accuracy independent of the platform used for obtaining the sequencing reads. In addition other sequencing platforms, such as the PacBio platform, make use of the same or a similar (strand displacement) DNA polymerase. The example shows that the DNA polymerase used for DNA-ball generation in MGI sequencing can progress through the linker moiety and it can therefore be reasonably expected that the method also works on other sequencing platforms, such as the PacBIO and Element Biosciences sequencing platforms.

TABLE 38
Validation of Read-pairs of the linked-adapter sequences between reads. R1 = Read 1, R2 = Read 2
and C = Consensus

| | Read-pair 1 | | | Read-pair 2 | | | Read-pair 3 | | | Read-pair 4 | | |
|---|---|---|---|---|---|---|---|---|---|---|---|---|
| | R 1 | R 2 | C | R 1 | R 2 | C | R 1 | R 2 | C | R 1 | R 2 | C |
| No. of | 0 | 3 | 0 | 0 | 1 | 0 | 0 | 6 | 0 | 1 | 0 | 0 |
| miscalls | | | | | | | | | | | | |

TABLE 39
Validation of Read-pairs of the linked-adapter sequences within
each of the single reads. R1 = Read 1 and R2 = Read 2.

| | Read-pair 1 | | Read-pair 2 | | Read-pair 3 | | Read-pair 4 | |
|---|---|---|---|---|---|---|---|---|
| | R 1 | R 2 | R 1 | R 2 | R 1 | R 2 | R 1 | R 2 |
| Number of | 0 | 2 | 0 | 1 | 0 | 3 | 1 | 0 |
| miscalls in the | | | | | | | | |
| first half of the | | | | | | | | |
| read | | | | | | | | |
| Number of | 0 | 1 | 0 | 0 | 0 | 3 | 0 | 0 |
| miscalls in the | | | | | | | | |
| second half of | | | | | | | | |
| the read | | | | | | | | |
| No. of | 0 | 0 | 0 | 0 | 0 | 0 | 0 | 0 |
| miscalls in | | | | | | | | |
| Consensus | | | | | | | | |

1. An at least partly double-stranded adapter, wherein the adapter comprises an open end and a closed end, and wherein the closed end is covalently closed by a linker, wherein said linker does not consist of deoxyribonucleotides.

2. An adapter according to claim 1, wherein the linker comprises a moiety of general formula (L1), with 5′ and 3′ used to represent sites of attachment to the nucleotides forming the end of the adapter: 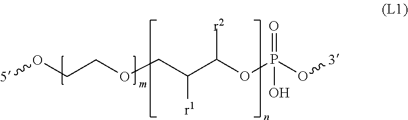
wherein
m is 0, 1, 2, 3, 4, 5, 6, 7, 8, 9, or 10;
n is 0 or 1;
r¹ and r² are each H, or together form a bridging moiety —(O)₀₋₁—(CH₂)₁₋₆—,
wherein preferably when n is 1, m is 0, or when m is not 0, n is 0,
and wherein preferably the linker is selected from the group consisting of C3 Spacer, Spacer 9, Spacer 18, or one or more 1′,2′-dideoxyriboses, wherein
the C3 Spacer is a moiety of general formula (L1), wherein m is 0, n is 1, r¹ is Hand r² is H,
the Spacer 9 is a moiety of general formula (L1), wherein m is 3 and n is 0,
the Spacer 18 is a moiety of general formula (L1), wherein m is 6 and n is 0, and
the 1′,2′-dideoxyribose is a moiety of general formula (L1), wherein m is 0, n is 1 and r¹ and r² together form bridging moiety —O—(CH₂)₂—.

3. The adapter according to claim 1, wherein the adapter comprises a staggered open end.

4. The adapter according to claim 1, wherein the adapter is formed by a single-stranded nucleic acid molecule comprising the linker.

5. The adapter according to claim 1, wherein the adapter comprises an identifier sequence.

6. A method for determining a sequence of interest in a double-stranded nucleic acid molecule, wherein the method comprises the steps of:
a) providing a sample comprising the double-stranded nucleic acid molecule and an at least partly double-stranded adapter, wherein the adapter comprises an open end and a closed end, and wherein the closed end is covalently closed by a linker, wherein said linker does not consist of deoxyribonucleotides;
b) ligating the adapter to an end of the double-stranded nucleic acid molecule, thereby generating a double-stranded nucleic acid molecule having one open end and one closed end;
c) sequencing both strands of at least part of the double-stranded nucleic acid molecule in a single sequencing reaction to generate a duplex read, wherein preferably the sequencing is performed on an amplification free sequencing platform; and
d) generating a consensus sequence from the duplex read to determine the sequence in the double-stranded nucleic acid molecule.

7. The method according to claim 6, wherein step b) comprises the (sub-)steps of:
b1) ligating the adapter to both ends of the double-stranded nucleic acid molecule, thereby closing both ends of the nucleic acid molecule;
b2) optionally exposing the sample to an exonuclease; and
b3) cleaving the closed double-stranded nucleic acid molecule, thereby generating the double-stranded nucleic acid molecule having one open end and one closed end.

8. The method according to claim 6, wherein the nucleic acid molecule is provided by fragmentation of a longer nucleic acid molecule, wherein the longer nucleic acid molecule is preferably a genomic nucleic acid molecule.

9. The method according to claim 8, wherein the fragmentation is performed by restriction endonuclease and/or site-directed endonuclease digestion.

10. The method according to claim 9, wherein the restriction enzyme and/or site-directed endonuclease creates a single-stranded overhang to the double-stranded nucleic acid molecule.

11. The method according to claim 6, wherein the nucleic acid molecule provided in step a) comprises a single-stranded overhang and wherein the adapter comprises an overhang that can be ligated to the overhang of the nucleic acid molecule.

12. The method according to claim 7, wherein the closed double-stranded nucleic acid molecule in step b3) is cleaved by a site-directed endonuclease or a restriction endonuclease, wherein preferably the site-directed endonuclease is an RNA-guided CRISPR nuclease or a TALENs.

13. A method according to claim 6, wherein the method is performed for a plurality of samples.

14. A method according to claim 6, wherein a sequencing adapter is ligated to the open end of the double-stranded nucleic acid after step b) and prior to step c).

15. A method according to claim 6, wherein the amplification free sequencing platform of step c) is nanopore sequencing.

16. The method according to claim 13, wherein the plurality of samples are pooled prior to step c).

17. The method according to claim 16, wherein nanopore selective sequencing.


SEQUENCE LISTING (42 sequences)

SEQ ID NO: 1 - protein, 1368 aa, synthetic construct [sequence omitted]

SEQ ID NO: 2 - DNA, 4104 bp, synthetic construct
  source 1..4104: mol_type = other DNA; note = Cas9 nt
atggataaaaaatatagcattggtctggatattggtaccaatagcgttggttgggcagttattaccgatgaatataaagttccgagcaaaaaatttaaagttctgggtaataccgatcgtcatagcattaaaaaaaatctgattggtgcactgctgtttgatagcggtgaaaccgcagaagcaacccgtctgaaacgtaccgcacgtcgtcgttatacccgtcgtaaaaatcgtatttgttatctgcaggaaatttttagcaatgaaatggcaaaagttgatgatagcttttttcatcgtctggaagaaagctttctggttgaagaagataaaaaacatgaacgtcatccgatttttggtaatattgttgatgaagttgcatatcatgaaaaatatccgaccatttatcatctgcgtaaaaaactggttgatagcaccgataaagcagatctgcgtctgatttatctggcactggcacatatgattaaatttcgtggtcattttctgattgaaggtgatctgaatccggataatagcgatgttgataaactgtttattcagctggttcagacctataatcagctgtttgaagaaaatccgattaatgcaagcggtgttgatgcaaaagcaattctgagcgcacgtctgagcaaaagccgtcgtctggaaaatctgattgcacagctgccgggtgaaaaaaaaaatggtctgtttggtaatctgattgcactgagcctgggtctgaccccgaattttaaaagcaattttgatctggcagaagatgcaaaactgcagctgagcaaagatacctatgatgatgatctggataatctgctggcacagattggtgatcagtatgcagatctgtttctggcagcaaaaaatctgagcgatgcaattctgctgagcgatattctgcgtgttaataccgaaattaccaaagcaccgctgagcgcaagcatgattaaacgttatgatgaacatcatcaggatctgaccctgctgaaagcactggttcgtcagcagctgccggaaaaatataaagaaattttttttgatcagagcaaaaatggttatgcaggttatattgatggtggtgcaagccaggaagaattttataaatttattaaaccgattctggaaaaaatggatggtaccgaagaactgctggttaaactgaatcgtgaagatctgctgcgtaaacagcgtacctttgataatggtagcattccgcatcagattcatctgggtgaactgcatgcaattctgcgtcgtcaggaagatttttatccgtttctgaaagataatcgtgaaaaaattgaaaaaattctgacctttcgtattccgtattatgttggtccgctggcacgtggtaatagccgttttgcatggatgacccgtaaaagcgaagaaaccattaccccgtggaattttgaagaagttgttgataaaggtgcaagcgcacagagctttattgaacgtatgaccaattttgataaaaatctgccgaatgaaaaagttctgccgaaacatagcctgctgtatgaatattttaccgtttataatgaactgaccaaagttaaatatgttaccgaaggtatgcgtaaaccggcatttctgagcggtgaacagaaaaaagcaattgttgatctgctgtttaaaaccaatcgtaaagttaccgttaaacagctgaaagaagattattttaaaaaaattgaatgttttgatagcgttgaaattagcggtgttgaagatcgttttaatgcaagcctgggtacctatcatgatctgctgaaaattattaaagataaagattttctggataatgaagaaaatgaagatattctggaagatattgttctgaccctgaccctgtttgaagatcgtgaaatgattgaagaacgtctgaaaacctatgcacatctgtttgatgataaagttatgaaacagctgaaacgtcgtcgttataccggttggggtcgtctgagccgtaaactgattaatggtattcgtgataaacagagcggtaaaaccattctggattttctgaaaagcgatggttttgcaaatcgtaattttatgcagctgattcatgatgatagcctgacctttaaagaagatattcagaaagcacaggttagcggtcagggtgatagcctgcatgaacatattgcaaatctggcaggtagcccggcaattaaaaaaggtattctgcagaccgttaaagttgttgatgaactggttaaagttatgggtcgtcataaaccggaaaatattgttattgaaatggcacgtgaaaatcagaccacccagaaaggtcagaaaaatagccgtgaacgtatgaaacgtattgaagaaggtattaaagaactgggtagccagattctgaaagaacatccggttgaaaatacccagctgcagaatgaaaaactgtatctgtattatctgcagaatggtcgtgatatgtatgttgatcaggaactggatattaatcgtctgagcgattatgatgttgatcatattgttccgcagagctttctgaaagatgatagcattgataataaagttctgacccgtagcgataaaaatcgtggtaaaagcgataatgttccgagcgaagaagttgttaaaaaaatgaaaaattattggcgtcagctgctgaatgcaaaactgattacccagcgtaaatttgataatctgaccaaagcagaacgtggtggtctgagcgaactggataaagcaggttttattaaacgtcagctggttgaaacccgtcagattaccaaacatgttgcacagattctggatagccgtatgaataccaaatatgatgaaaatgataaactgattcgtgaagttaaagttattaccctgaaaagcaaactggttagcgattttcgtaaagattttcagttttataaagttcgtgaaattaataattatcatcatgcacatgatgcatatctgaatgcagttgttggtaccgcactgattaaaaaatatccgaaactggaaagcgaatttgtttatggtgattataaagtttatgatgttcgtaaaatgattgcaaaaagcgaacaggaaattggtaaagcaaccgcaaaatattttttttatagcaatattatgaatttttttaaaaccgaaattaccctggcaaatggtgaaattcgtaaacgtccgctgattgaaaccaatggtgaaaccggtgaaattgtttgggataaaggtcgtgattttgcaaccgttcgtaaagttctgagcatgccgcaggttaatattgttaaaaaaaccgaagttcagaccggtggttttagcaaagaaagcattctgccgaaacgtaatagcgataaactgattgcacgtaaaaaagattgggatccgaaaaaatatggtggttttgatagcccgaccgttgcatatagcgttctggttgttgcaaaagttgaaaaaggtaaaagcaaaaaactgaaaagcgttaaagaactgctgggtattaccattatggaacgtagcagctttgaaaaaaatccgattgattttctggaagcaaaaggttataaagaagttaaaaaagatctgattattaaactgccgaaatatagcctgtttgaactggaaaatggtcgtaaacgtatgctggcaagcgcaggtgaactgcagaaaggtaatgaactggcactgccgagcaaatatgttaattttctgtatctggcaagccattatgaaaaactgaaaggtagcccggaagataatgaacagaaacagctgtttgttgaacagcataaacattatctggatgaaattattgaacagattagcgaatttagcaaacgtgttattctggcagatgcaaatctggataaagttctgagcgcatataataaacatcgtgataaaccgattcgtgaacaggcagaaaatattattcatctgtttaccctgaccaatctgggtgcaccggcagcatttaaatattttgataccaccattgatcgtaaacgttataccagcaccaaagaagttctggatgcaaccctgattcatcagagcattaccggtctgtatgaaacccgtattgatctgagccagctgggtggtgat

SEQ ID NO: 3 - protein, 1082 aa, synthetic construct
  source 1..1082: note = Cas9 no. 2 a.a.
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 4 - protein, 1300 aa, synthetic construct
  source 1..1300: note = Cpf1 a.a.
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 5 - DNA, 3900 bp, synthetic construct
  source 1..3900: mol_type = other DNA; note = Cpf1 nt
atgagcatttatcaggaatttgttaataaatatagcctgagcaaaaccctgcgttttgaactgattccgcagggtaaaaccctggaaaatattaaagcacgtggtctgattctggatgatgaaaaacgtgcaaaagattataaaaaagcaaaacagattattgataaatatcatcagttttttattgaagaaattctgagcagcgtttgtattagcgaagatctgctgcagaattatagcgatgtttattttaaactgaaaaaaagcgatgatgataatctgcagaaagattttaaaagcgcaaaagataccattaaaaaacagattagcgaatatattaaagatagcgaaaaatttaaaaatctgtttaatcagaatctgattgatgcaaaaaaaggtcaggaaagcgatctgattctgtggctgaaacagagcaaagataatggtattgaactgtttaaagcaaatagcgatattaccgatattgatgaagcactggaaattattaaaagctttaaaggttggaccacctattttaaaggttttcatgaaaatcgtaaaaatgtttatagcagcaatgatattccgaccagcattatttatcgtattgttgatgataatctgccgaaatttctggaaaataaagcaaaatatgaaagcctgaaagataaagcaccggaagcaattaattatgaacagattaaaaaagatctggcagaagaactgacctttgatattgattataaaaccagcgaagttaatcagcgtgtttttagcctggatgaagtttttgaaattgcaaattttaataattatctgaatcagagcggtattaccaaatttaataccattattggtggtaaatttgttaatggtgaaaataccaaacgtaaaggtattaatgaatatattaatctgtatagccagcagattaatgataaaaccctgaaaaaatataaaatgagcgttctgtttaaacagattctgagcgataccgaaagcaaaagctttgttattgataaactggaagatgatagcgatgttgttaccaccatgcagagcttttatgaacagattgcagcatttaaaaccgttgaagaaaaaagcattaaagaaaccctgagcctgctgtttgatgatctgaaagcacagaaactggatctgagcaaaatttattttaaaaatgataaaagcctgaccgatctgagccagcaggtttttgatgattatagcgttattggtaccgcagttctggaatatattacccagcagattgcaccgaaaaatctggataatccgagcaaaaaagaacaggaactgattgcaaaaaaaaccgaaaaagcaaaatatctgagcctggaaaccattaaactggcactggaagaatttaataaacatcgtgatattgataaacagtgtcgttttgaagaaattctggcaaattttgcagcaattccgatgatttttgatgaaattgcacagaataaagataatctggcacagattagcattaaatatcagaatcagggtaaaaaagatctgctgcaggcaagcgcagaagatgatgttaaagcaattaaagatctgctggatcagaccaataatctgctgcataaactgaaaatttttcatattagccagagcgaagataaagcaaatattctggataaagatgaacatttttatctggtttttgaagaatgttattttgaactggcaaatattgttccgctgtataataaaattcgtaattatattacccagaaaccgtatagcgatgaaaaatttaaactgaattttgaaaatagcaccctggcaaatggttgggataaaaataaagaaccggataataccgcaattctgtttattaaagatgataaatattatctgggtgttatgaataaaaaaaataataaaatttttgatgataaagcaattaaagaaaataaaggtgaaggttataaaaaaattgtttataaactgctgccgggtgcaaataaaatgctgccgaaagttttttttagcgcaaaaagcattaaattttataatccgagcgaagatattctgcgtattcgtaatcatagcacccataccaaaaatggtagcccgcagaaaggttatgaaaaatttgaatttaatattgaagattgtcgtaaatttattgatttttataaacagagcattagcaaacatccggaatggaaagattttggttttcgttttagcgatacccagcgttataatagcattgatgaattttatcgtgaagttgaaaatcagggttataaactgacctttgaaaatattagcgaaagctatattgatagcgttgttaatcagggtaaactgtatctgtttcagatttataataaagattttagcgcatatagcaaaggtcgtccgaatctgcataccctgtattggaaagcactgtttgatgaacgtaatctgcaggatgttgtttataaactgaatggtgaagcagaactgttttatcgtaaacagagcattccgaaaaaaattacccatccggcaaaagaagcaattgcaaataaaaataaagataatccgaaaaaagaaagcgtttttgaatatgatctgattaaagataaacgttttaccgaagataaatttttttttcattgtccgattaccattaattttaaaagcagcggtgcaaataaatttaatgatgaaattaatctgctgctgaaagaaaaagcaaatgatgttcatattctgagcattgatcgtggtgaacgtcatctggcatattataccctggttgatggtaaaggtaatattattaaacaggatacctttaatattattggtaatgatcgtatgaaaaccaattatcatgataaactggcagcaattgaaaaagatcgtgatagcgcacgtaaagattggaaaaaaattaataatattaaagaaatgaaagaaggttatctgagccaggttgttcatgaaattgcaaaactggttattgaatataatgcaattgttgtttttgaagatctgaattttggttttaaacgtggtcgttttaaagttgaaaaacaggtttatcagaaactggaaaaaatgctgattgaaaaactgaattatctggtttttaaagataatgaatttgataaaaccggtggtgttctgcgtgcatatcagctgaccgcaccgtttgaaacctttaaaaaaatgggtaaacagaccggtattatttattatgttccggcaggttttaccagcaaaatttgtccggttaccggttttgttaatcagctgtatccgaaatatgaaagcgttagcaaaagccaggaattttttagcaaatttgataaaatttgttataatctggataaaggttattttgaatttagctttgattataaaaattttggtgataaagcagcaaaaggtaaatggaccattgcaagctttggtagccgtctgattaattttcgtaatagcgataaaaatcataattgggatacccgtgaagtttatccgaccaaagaactggaaaaactgctgaaagattatagcattgaatatggtcatggtgaatgtattaaagcagcaatttgtggtgaaagcgataaaaaattttttgcaaaactgaccagcgttctgaataccattctgcagatgcgtaatagcaaaaccggtaccgaactggattatctgattagcccggttgcagatgttaatggtaatttttttgatagccgtcaggcaccgaaaaatatgccgcaggatgcagatgcaaatggtgcatatcatattggtctgaaaggtctgatgctgctgggtcgtattaaaaataatcaggaaggtaaaaaactgaatctggttattaaaaatgaagaatattttgaatttgttcagaatcgtaataat

SEQ ID NO: 6 - protein, 1263 aa, synthetic construct
  source 1..1263: note = Mad7 a.a. no. 1
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 7 - protein, 1274 aa, synthetic construct
  source 1..1274: note = Mad7 a.a. no. 2
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLSGGSPKKKRKV

SEQ ID NO: 8 - protein, 1602 aa, synthetic construct
  source 1..1602: note = FokI-G4S-dCas9-NLS-3xFLAG
MQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDK

SEQ ID NO: 9 - protein, 1401 aa, synthetic construct
  source 1..1401: note = Nls-FokI-dCas9-nls
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDK

SEQ ID NO: 10 - protein, 1787 aa, synthetic construct
  source 1..1787: note = Csy4-T2A-Clo051-G4Slinker-dCas9
MGDHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWFGTEGRGSLLTCGDVEENPGPMPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - DNA, 41 bp, synthetic construct
  source 1..41: mol_type = other DNA
aaggttaacacaaagacaccgacaactttcttcagcacctc

SEQ ID NO: 12 - DNA, 45 bp, synthetic construct
  source 1..45: mol_type = other DNA
agctgaggtgctgaagaaagttgtcggtgtctttgtgttaacctt

SEQ ID NO: 13 - DNA, 41 bp, synthetic construct
  source 1..41: mol_type = other DNA
aaggttaaacagacgactacaaacggaatcgacagcacctc

SEQ ID NO: 14 - DNA, 45 bp, synthetic construct
  source 1..45: mol_type = other DNA
agctgaggtgctgtcgattccgtttgtagtcgtctgtttaacctt

SEQ ID NO: 15 - DNA, 41 bp, synthetic construct
  source 1..41: mol_type = other DNA
aaggttaacctggtaactgggacacaagactccagcacctc

SEQ ID NO: 16 - DNA, 45 bp, synthetic construct
  source 1..45: mol_type = other DNA
agctgaggtgctggagtcttgtgtcccagttaccaggttaacctt

SEQ ID NO: 17 - DNA, 41 bp, synthetic construct
  source 1..41: mol_type = other DNA
aaggttaatagggaaacacgatagaatccgaacagcacctc

SEQ ID NO: 18 - DNA, 45 bp, synthetic construct
  source 1..45: mol_type = other DNA
agctgaggtgctgttcggattctatcgtgtttccctattaacctt

SEQ ID NO: 19 - DNA, 41 bp, synthetic construct
  source 1..41: mol_type = other DNA
aaggttaaaaggttacacaaaccctggacaagcagcacctc

SEQ ID NO: 20 - DNA, 45 bp, synthetic construct
  source 1..45: mol_type = other DNA
agctgaggtgctgcttgtccagggtttgtgtaaccttttaacctt

SEQ ID NO: 21 - DNA, 41 bp, synthetic construct
  source 1..41: mol_type = other DNA
aaggttaagactactttctgcctttgcgagaacagcacctc

SEQ ID NO: 22 - DNA, 45 bp, synthetic construct
  source 1..45: mol_type = other DNA
agctgaggtgctgttctcgcaaaggcagaaagtagtcttaacctt

SEQ ID NO: 23 - DNA, 27 bp, synthetic construct
  source 1..27: mol_type = other DNA
  misc_feature 27: note = T on position 27 is linked to A on position 1 of SEQ ID NO: 24 via a C3 spacer
gcaatacgtaactgaacgaagtacatt

SEQ ID NO: 24 - DNA, 28 bp, synthetic construct
  source 1..28: mol_type = other DNA
  misc_feature 1: note = A on position 1 is linked to T on position 27 of SEQ ID NO: 23 via a C3 linker
aatgtacttcgttcagttacgtattgct

SEQ ID NO: 25 - DNA, 27 bp, synthetic construct
  source 1..27: mol_type = other DNA
  misc_feature 27: note = T on position 27 is linked to A on position 1 of SEQ ID NO: 26 via a C9 spacer
gcaatacgtaactgaacgaagtacatt

SEQ ID NO: 26 - DNA, 28 bp, synthetic construct
  source 1..28: mol_type = other DNA
  misc_feature 1: note = A on position 1 is linked to T on position 27 of SEQ ID NO: 25 via a C9 spacer
aatgtacttcgttcagttacgtattgct

SEQ ID NO: 27 - DNA, 27 bp, synthetic construct
  source 1..27: mol_type = other DNA
  misc_feature 27: note = T on position 27 is linked to A on position 1 of SEQ ID NO: 28 via a C18 spacer
gcaatacgtaactgaacgaagtacatt

SEQ ID NO: 28 - DNA, 28 bp, synthetic construct
  source 1..28: mol_type = other DNA
  misc_feature 1: note = A on position 1 is linked to T on position 27 of SEQ ID NO: 27 via a C18 spacer
aatgtacttcgttcagttacgtattgct

SEQ ID NO: 29 - DNA, 27 bp, synthetic construct
  source 1..27: mol_type = other DNA
  misc_feature 27: note = T on position 27 is linked to A on position 1 of SEQ ID NO: 30 via a 1,2-dideoxyribose spacer
gcaatacgtaactgaacgaagtacatt

SEQ ID NO: 30 - DNA, 28 bp, synthetic construct
  source 1..28: mol_type = other DNA
  misc_feature 1: note = A on position 1 is linked to T on position 27 of SEQ ID NO: 29 via a 1,2-dideoxyribose spacer
aatgtacttcgttcagttacgtattgct

SEQ ID NO: 31 - DNA, 27 bp, synthetic construct
  source 1..27: mol_type = other DNA
  misc_feature 27: note = T on position 27 is linked to A on position 1 of SEQ ID NO: 32 via a 3 moieties of a 1,2-dideoxyribose spacer
gcaatacgtaactgaacgaagtacatt

SEQ ID NO: 32 - DNA, 28 bp, synthetic construct
  source 1..28: mol_type = other DNA
  misc_feature 1: note = A on position 1 is linked to T on position 27 of SEQ ID NO: 31 via 3 moieties of a 1,2-dideoxyribose spacer
aatgtacttcgttcagttacgtattgct

SEQ ID NO: 33 - DNA, 27 bp, synthetic construct
  source 1..27: mol_type = other DNA
  misc_feature 27: note = T on position 27 is linked to A on position 1 of SEQ ID NO: 34 via 5 moieties of a 1,2-dideoxyribose spacer
gcaatacgtaactgaacgaagtacatt

SEQ ID NO: 34 - DNA, 28 bp, synthetic construct
  source 1..28: mol_type = other DNA
  misc_feature 1: note = A on position 1 is linked to T on position 27 of SEQ ID NO: 33 via 5 moieties of a 1,2-dideoxyribose spacer
aatgtacttcgttcagttacgtattgct

SEQ ID NO: 35 - RNA, 122 nt, synthetic construct
  source 1..122: mol_type = other RNA
taatacgactcactataggctgcttcgtttatgcgctgggttttagagctagaaatagcaagttaaaataaggctagtccgttatcaacttgaaaaagtggcaccgagtcggtgcttttttt

SEQ ID NO: 36 - RNA, 122 nt, synthetic construct
  source 1..122: mol_type = other RNA
taatacgactcactataggcgccacgtctgcctccaccggttttagagctagaaatagcaagttaaaataaggctagtccgttatcaacttgaaaaagtggcaccgagtcggtgcttttttt

SEQ ID NO: 37 - RNA, 122 nt, synthetic construct
  source 1..122: mol_type = other RNA
taatacgactcactatagggcgctttctactcgtgatttgttttagagctagaaatagcaagttaaaataaggctagtccgttatcaacttgaaaaagtggcaccgagtcggtgcttttttt

SEQ ID NO: 38 - RNA, 122 nt, synthetic construct
  source 1..122: mol_type = other RNA
taatacgactcactataggatttacaatatcgtcctgttgttttagagctagaaatagcaagttaaaataaggctagtccgttatcaacttgaaaaagtggcaccgagtcggtgcttttttt

SEQ ID NO: 39 - DNA, 27 bp, synthetic construct
  source 1..27: mol_type = other DNA
gcaatacgtaactgaacgaagtacatt

SEQ ID NO: 40 - DNA, 36 bp, synthetic construct
  source 1..36: mol_type = other DNA
aatgtacttcgttcagttacgtattgcgcggccgct

SEQ ID NO: 41 - DNA, 58 bp, synthetic construct
  source 1..58: mol_type = other DNA
agtcggaggccaagcggtcttaggaagacaataggtccgatcaactccttggctcaca

SEQ ID NO: 42 - DNA, 25 bp, synthetic construct
  source 1..25: mol_type = other DNA
gaacgacatggctacgatccgactt